(19) United States
(12) Patent Application Publication
(10) Pub. No.: US 2019/0002417 A1
(43) Pub. Date: Jan. 3, 2019

(54) MATERIALS FOR ELECTRONIC DEVICES

(71) Applicant: Merck Patent GmbH, Darmstadt (DE)

(72) Inventors: Stefan Riedmueller, Frankfurt am Main (DE); Oliver Kaufhold, Darmstadt (DE); Sebastian Meyer, Aschaffenburg (DE)

(73) Assignee: Merck Patent GmbH, Darmstadt (DE)

(21) Appl. No.: 16/064,028

(22) PCT Filed: Nov. 28, 2016
(86) PCT No.: PCT/EP2016/002011

§ 371 Date: Jun. 20, 2018

(30) Foreign Application Priority Data
Dec. 21, 2015 (EP) 15201728.1

(51) Int. Cl. C07D 265/38; C07D 223/22; C07D 209/94; C07D 221/18; C07D 279/36; H01L 51/00

(52) U.S. Cl. CPC C07D 265/38; C07D 223/22; C07D 209/94; C07D 221/18; H01L 51/5056; H01L 51/0072; H01L 51/0056; H01L 51/0035; C07D 279/36

(57) ABSTRACT

The present invention relates to compounds according to formula (I), which are suitable for use in electronic devices, preferably organic electroluminescent devices.

The present invention relates to monobenzoindenofluorene compounds, to the use of the compounds in electronic devices, to electronic devices comprising the compounds, and to processes for preparing the compounds.

There is currently an interest in developing compounds with which improved properties of electronic devices can be achieved in one or more relevant aspects, for example power efficiency, lifetime and color coordinates of the light emitted.

The term “electronic device” is understood according to the present application to mean electronic devices in general that contain organic materials. More particularly, these are understood to mean organic integrated circuits (OICs), organic field-effect transistors (OFETs), organic thin-film transistors (OTFTs), organic light-emitting transistors (OLETs), organic solar cells (OSCs), organic optical detectors, organic photoreceptors, organic field-quench devices (OFQDs), organic light-emitting electrochemical cells (OLECs), organic laser diodes (O-lasers) and organic electroluminescent devices (OLEDs).

Of particular interest is the provision of compounds for use in the latter electronic devices referred to as OLEDs. The general structure of OLEDs and the way in which they work is known to those skilled in the art and described, inter alia, in U.S. Pat. No. 4,539,507, U.S. Pat. No. 5,151,629, EP 0676461 and WO 1998/27136.

With regard to the performance data of OLEDs, further improvements are still required, especially with regard to broad commercial use, for example in displays or as light sources. Of particular significance in this connection are the lifetime, the efficiency and the operating voltage of the OLEDs, and the color values achieved. Especially in the case of blue-emitting OLEDs, there is potential for improvement with regard to the lifetime of the devices.

Of great significance in this connection is the choice of compound which is used as emitting compound in the OLED.

For this purpose, the prior art discloses a multitude of compounds, especially arylamines having one or more fused aryl groups.

Mention should be made here by way of example of the compounds disclosed in WO 2008/006449, which are based on an indenofluorene skeleton in which one of the phenyl groups has been extended to form a larger aryl group, for example to form a naphthyl group.

The compounds disclosed in the abovementioned applications are valuable functional compounds, but they can still be improved with regard to particular aspects. More particularly, owing to the ever rising demands, there is continuous need for improvement in relation to power efficiency and lifetime.

It has been found that, surprisingly, the novel compounds defined hereinafter which have an N-heterocyclic group bonded to a monobenzoindenofluorene base skeleton bring about improvements in the power efficiency and the lifetime of the OLEDs.

The present application thus provides compounds of formula (I)

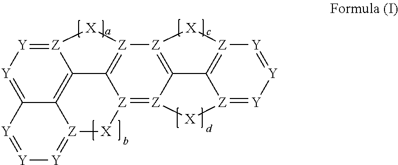

where the variables that occur are as follows:
Y is the same or different at each instance and is selected from CR¹ and N;
Z is C if an X bridge is bonded to the Z group, and is Y if no X bridge is bonded to the Z group;
X is the same or different at each instance and is a bivalent bridge selected from C(R¹)₂ and Si(R¹)₂;
R¹ is the same or different at each instance and is selected from H, D, F, C(═O)R², CN, Si(R²)₃, N(R²)₂, P(═O)(R²)₂, OR², S(═O)R², S(═O)₂R², straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R¹ radicals may be joined to one another and may form a ring; where the alkyl, alkoxy, alkenyl and alkynyl groups mentioned and the aromatic ring systems and heteroaromatic ring systems mentioned may each be substituted by one or more R² radicals; and where one or more CH₂ groups in the alkyl, alkoxy, alkenyl and alkynyl groups mentioned may be replaced by —R²C═CR²—, —C≡C—, Si(R²)₂, C═O, C═NR², —C(═O)O—, C(═O)NR²—, NR², P(═O)(R²), —O—, —S—, SO or SO₂;
R² is the same or different at each instance and is selected from H, D, F, CN, alkyl groups having 1 to 20 carbon atoms, aromatic ring systems having 6 to 40 aromatic ring atoms and heteroaromatic ring systems having 5 to 40 aromatic ring atoms; where two or more R² radicals may be joined to one another and may form a ring; and where the alkyl groups, aromatic ring systems and heteroaromatic ring systems mentioned may be substituted by F or CN;
a, b, c, d is the same or different at each instance and is 0 or 1, with the proviso that a+b=1 and c+d=1, where a=0, b=0, c=0 and d=0 respectively mean that the corresponding X bridge is absent;
   where there is at least one Y in which a unit of the formula (N) is bonded instead of R¹,

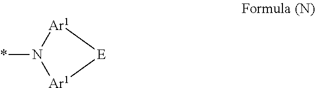

in which the bond identified by an asterisk marks the bond to Y and in which, in addition:
Ar¹ is the same or different at each instance and is selected from aromatic ring systems which have 6 to 30 aromatic ring atoms and may be substituted by one or more R¹ radicals, and heteroaromatic ring systems which have 5 to 30 aromatic ring atoms and may be substituted by one or more R¹ radicals;
E is a single bond or a divalent group selected from optionally R¹-substituted arylene groups, optionally R¹-substituted heteroarylene groups, B(R¹), C(R¹)₂, C(R¹)═C(R¹), C≡C, Si(R¹)₂, C═O, C═NR¹, C═C(R¹)₂, O, S, S═O, SO₂, N(R¹), P(R¹) and P(═O)R¹, or combinations of 2, 3 or 4 identical or different groups among these.

An aryl group in the context of this invention contains 6 to 40 aromatic ring atoms of which none is a heteroatom. An aryl group in the context of this invention is understood to mean either a simple aromatic cycle, i.e. benzene, or a fused aromatic polycycle, for example naphthalene, phenanthrene or anthracene. A fused aromatic polycycle in the context of the present application consists of two or more simple aromatic cycles fused to one another. Fusion between cycles is understood here to mean that the cycles share at least one edge with one another.

A heteroaryl group in the context of this invention contains 5 to 40 aromatic ring atoms of which at least one is a heteroatom. The heteroatoms of the heteroaryl group are preferably selected from N, O and S. A heteroaryl group in the context of this invention is understood to mean either a simple heteroaromatic cycle, for example pyridine, pyrimidine or thiophene, or a fused heteroaromatic polycycle, for example quinoline or carbazole. A fused heteroaromatic polycycle in the context of the present application consists of two or more simple heteroaromatic cycles fused to one another. Fusion between cycles is understood here to mean that the cycles share at least one edge with one another.

An arylene group and a heteroarylene group are correspondingly understood to mean the divalent units respectively derived from an aryl group and a heteroaryl group.

An aryl or heteroaryl group, each of which may be substituted by the abovementioned radicals and which may be joined to the aromatic or heteroaromatic system via any desired positions, is especially understood to mean groups derived from benzene, naphthalene, anthracene, phenanthrene, pyrene, dihydropyrene, chrysene, perylene, triphenylene, fluoranthene, benzanthracene, benzophenanthrene, tetracene, pentacene, benzopyrene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, pyrazine, phenazine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

An aromatic ring system in the context of this invention contains 6 to 40 carbon atoms in the ring system and does not include any heteroatoms as aromatic ring atoms. An aromatic ring system in the context of this invention therefore does not contain any heteroaryl groups. An aromatic ring system in the context of this invention shall be understood to mean a system which does not necessarily contain only aryl groups but in which it is also possible for a plurality of aryl groups to be bonded by a single bond or by a non-aromatic unit, for example one or more optionally substituted C, Si, N, O or S atoms. In this case, the non-aromatic unit comprises preferably less than 10% of the atoms other than H, based on the total number of atoms other than H in the system. For example, systems such as 9,9′-spirobifluorene, 9,9′-diarylfluorene, triarylamine, diaryl ethers and stilbene are also to be regarded as aromatic ring systems in the context of this invention, and likewise systems in which two or more aryl groups are joined, for example, by a linear or cyclic alkyl, alkenyl or alkynyl group or by a silyl group. In addition, systems in which two or more aryl groups are joined to one another via single bonds are also regarded as aromatic ring systems in the context of this invention, for example systems such as biphenyl and terphenyl.

A heteroaromatic ring system in the context of this invention contains 5 to 40 aromatic ring atoms, at least one of which is a heteroatom. The heteroatoms of the heteroaromatic ring system are preferably selected from N, O and/or S. A heteroaromatic ring system corresponds to the abovementioned definition of an aromatic ring system, but has at least one heteroatom as one of the aromatic ring atoms. In this way, it differs from an aromatic ring system in the sense of the definition of the present application, which, according to this definition, cannot contain any heteroatom as aromatic ring atom.

An aromatic ring system having 6 to 40 aromatic ring atoms or a heteroaromatic ring system having 5 to 40 aromatic ring atoms is especially understood to mean groups derived from the groups mentioned above under aryl groups and heteroaryl groups, and from biphenyl, terphenyl, quaterphenyl, fluorene, spirobifluorene, dihydrophenanthrene, dihydropyrene, tetrahydropyrene, indenofluorene, truxene, isotruxene, spirotruxene, spiroisotruxene, indenocarbazole, or from combinations of these groups.

In the context of the present invention, a straight-chain alkyl group having 1 to 20 carbon atoms and a branched or cyclic alkyl group having 3 to 20 carbon atoms and an alkenyl or alkynyl group having 2 to 40 carbon atoms in which individual hydrogen atoms or CH₂ groups may also be substituted by the groups mentioned above in the definition of the radicals are preferably understood to mean the methyl, ethyl, n-propyl, i-propyl, n-butyl, i-butyl, s-butyl, t-butyl, 2-methylbutyl, n-pentyl, s-pentyl, cyclopentyl, neopentyl, n-hexyl, cyclohexyl, neohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl radicals.

An alkoxy or thioalkyl group having 1 to 20 carbon atoms in which individual hydrogen atoms or CH₂ groups may also be replaced by the groups mentioned above in the definition of the radicals is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy, 2,2,2-trifluoroethoxy, methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio.

The wording that two or more radicals together may form a ring, in the context of the present application, shall be understood to mean, inter alia, that the two radicals are joined to one another by a chemical bond. In addition, however, the abovementioned wording shall also be understood to mean that, if one of the two radicals is hydrogen, the second radical binds to the position to which the hydrogen atom was bonded, forming a ring.

Preferably in the compound of the formula (I), there is exactly one Y in which a unit of the formula (N) is bonded instead of R¹.

In units of the formula (N), Ar¹ is preferably the same or different at each instance and is selected from phenyl, naphthyl, phenanthrenyl, biphenyl, terphenyl, quaterphenyl, fluorenyl, spirobifluorenyl, indenofluorenyl, carbazolyl, dibenzothiophenyl, dibenzofuranyl, benzofuranyl, benzothiophenyl, indolyl, triazinyl, pyrimidinyl, pyridyl and pyridazinyl, where each of the groups mentioned may be substituted by one or more R¹ radicals.

More preferably, in formula (N), Art is the same or different at each instance and is selected from phenyl, biphenyl, terphenyl, fluorenyl and carbazolyl, where the groups mentioned may each be substituted by one or more R¹ radicals.

In a preferred embodiment, Art is selected identically at each instance.

Preferably, E in formula (N) is the same or different at each instance and is selected from a single bond, optionally R¹-substituted arylene groups, optionally R¹-substituted heteroarylene groups and the following divalent groups:

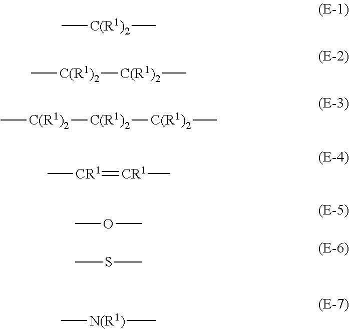

where the variables that occur are as defined above.

The optionally R¹-substituted arylene groups are preferably optionally R¹-substituted phenylene groups, more preferably optionally R¹-substituted 1,3-phenylene groups.

Preferred embodiments of the formula (N) correspond to the following formulae:

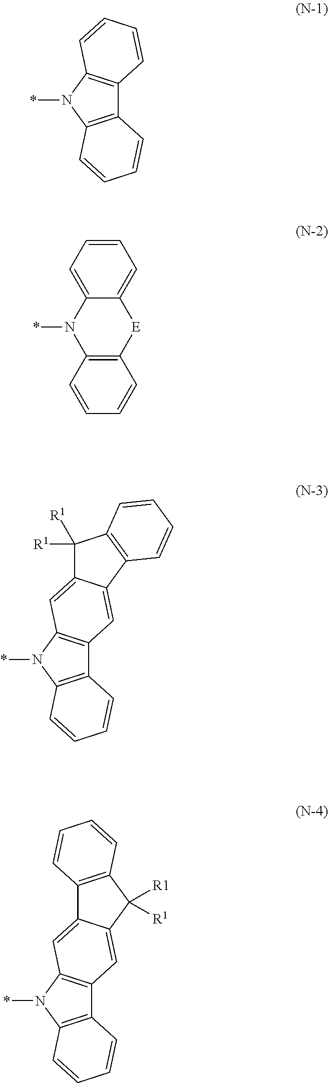

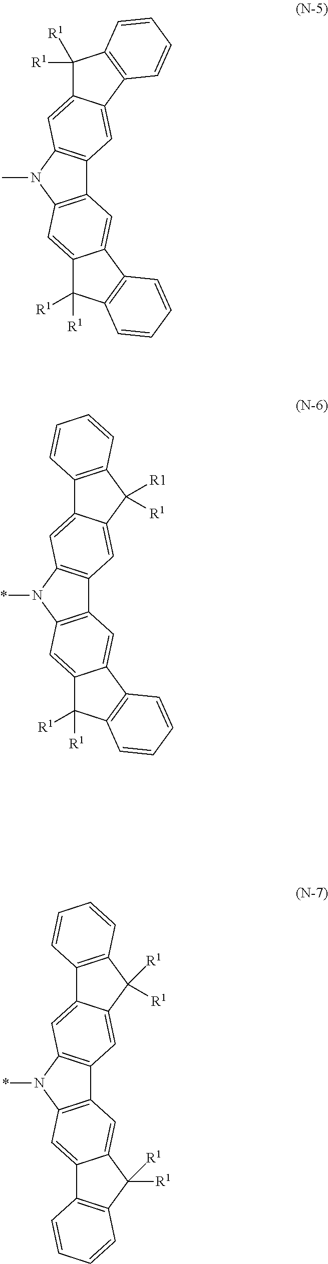

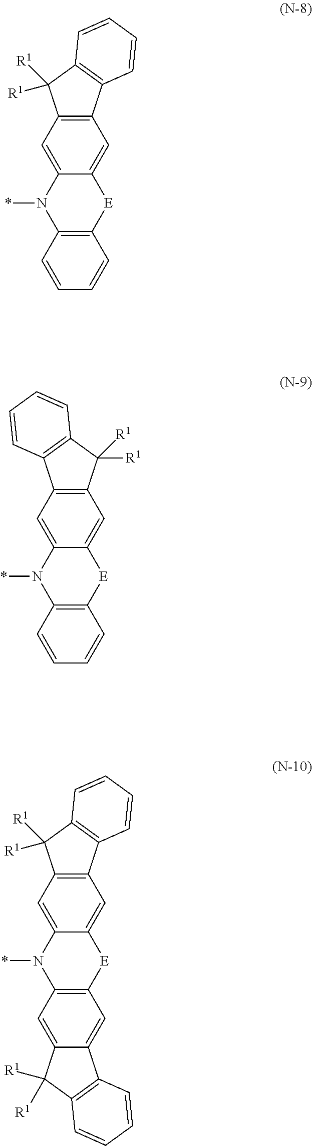

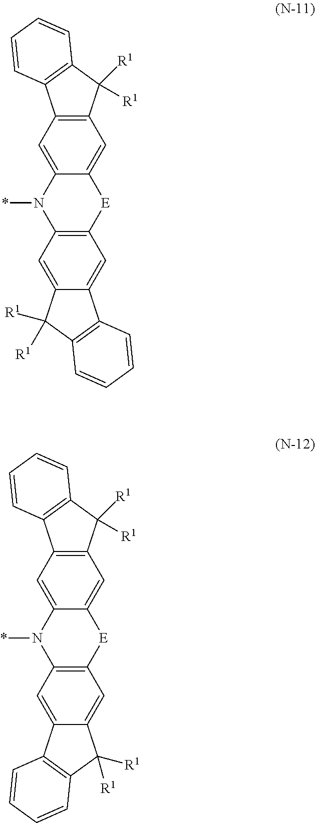

where the groups that occur are as defined above, and where the units of the formula (N) may be substituted at positions shown as unsubstituted by R¹ radicals.

Preferably, E in the units of the formula (N) is selected from optionally R¹-substituted arylene groups, optionally R¹-substituted heteroarylene groups and the divalent E-1 to E-7 groups.

Preferably, not more than three Y groups per aromatic six-membered ring are N, more preferably not more than 2. Most preferably, all Y groups are CR¹.

Preferably, X is C(R¹)₂.

Preferably, R¹ is the same or different at each instance and is selected from H, D, F, CN, Si(R²)₃, N(R²)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms, where the alkyl and alkoxy groups mentioned, the aromatic ring systems mentioned and the heteroaromatic ring systems mentioned may each be substituted by one or more R² radicals; and where one or more CH₂ groups in the alkyl or alkoxy groups mentioned may be replaced by —C≡C—, —R²C═CR²—, Si(R²)₂, C═O, C═NR², —NR²—, —O—, —S—, —C(═O)O— or —C(═O)NR²—.

It is further preferable that R¹ radicals in X units are the same or different at each instance and are selected from H, D, F, CN, straight-chain alkyl groups having 1 to 12 carbon atoms, branched or cyclic alkyl groups having 3 to 12 carbon atoms, aromatic ring systems having 6 to 20 aromatic ring atoms, and heteroaromatic ring systems having 5 to 20 aromatic ring atoms, where the alkyl groups mentioned, the aromatic ring systems mentioned and the heteroaromatic ring systems mentioned may each be substituted by one or more R² radicals.

More preferably, R¹ radicals in X units are the same or different at each instance and are selected from straight-chain alkyl groups having 1 to 12 carbon atoms, branched or cyclic alkyl groups having 3 to 12 carbon atoms and aromatic ring systems having 6 to 20 aromatic ring atoms; where the alkyl groups mentioned and the aromatic ring systems mentioned may each be substituted by one or more R² radicals.

R¹ radicals in Y units are preferably the same or different at each instance and are selected from H, D, F, CN, straight-chain alkyl groups having 1 to 20 carbon atoms, branched or cyclic alkyl groups having 3 to 20 carbon atoms, aromatic ring systems having 6 to 40 aromatic ring atoms, and heteroaromatic ring systems having 5 to 40 aromatic ring atoms, where the alkyl groups mentioned, the aromatic ring systems mentioned and the heteroaromatic ring systems mentioned may each be substituted by one or more R² radicals.

More preferably, R¹ radicals in Y units are H.

Preferred embodiments of the formula (I) correspond to the following formulae (I-1) to (I-4):

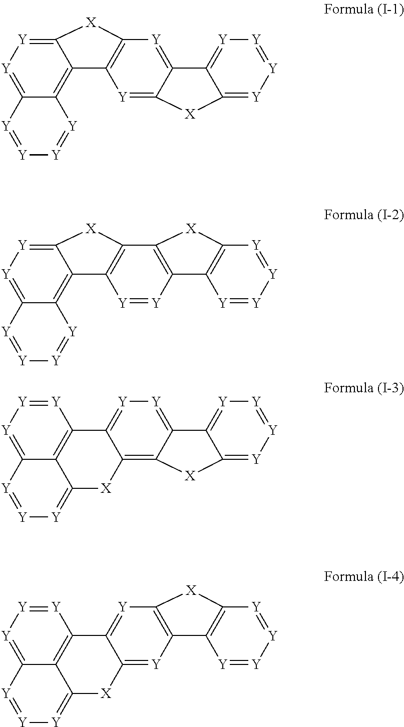

where the variables that occur are as defined above, and where there is at least one Yin which a unit of the formula (N), as defined above, is bonded instead of R¹.

Among the formulae (I-1) to (I-4), preference is given to formula (I-1).

Preference is further given to the combination of the preferred formulae for the base skeleton of formulae (I-1) to (I-4), especially of the formula (I-1), with one of the preferred formulae for the N group according to formulae (N-1) to (N-4).

Particularly preferred embodiments of the formula (I) correspond to the following formulae (I-1-1) to (I-4-1):

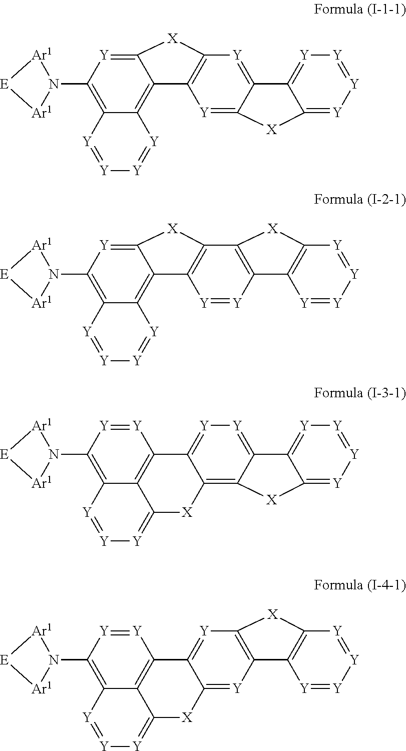

where the variables that occur are as defined above.

Among the formulae (I-1-1) to (I-4-1), preference is given to formula (I-1-1).

Preference is further given to the combination of the preferred formulae for the base skeleton of formulae (I-1-1) to (I-4-1), especially of the formula (I-1-1), with one of the preferred formulae for the N group according to formulae (N-1) to (N-4).

Examples of compounds of the formula (I) are depicted in the following table:

| | |
|---|---|
| 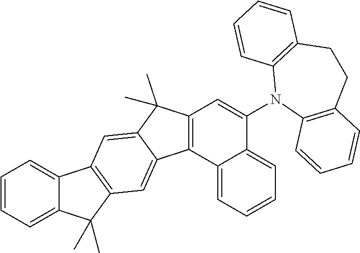 [SMILES: CC1(C)c2ccccc2-c2cc3c(cc21)-c1c(cc(N2c4ccccc4CCc4ccccc42)c2ccccc12)C3(C)C] | (1) |
| 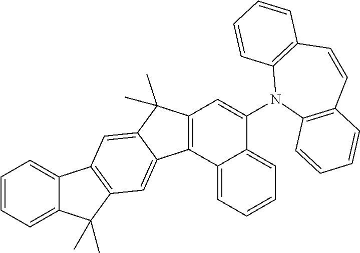 [SMILES: CC1(C)c2ccccc2-c2cc3c(cc21)-c1c(cc(N2c4ccccc4C=Cc4ccccc42)c2ccccc12)C3(C)C] | (2) |
| 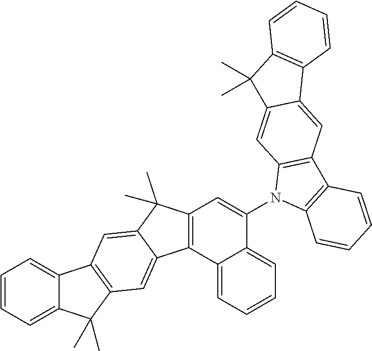 [SMILES: CC1(C)c2ccccc2-c2cc3c(cc21)-c1c(cc(-n2c4ccccc4c4cc5c(cc42)C(C)(C)c2ccccc2-5)c2ccccc12)C3(C)C] | (3) |
| 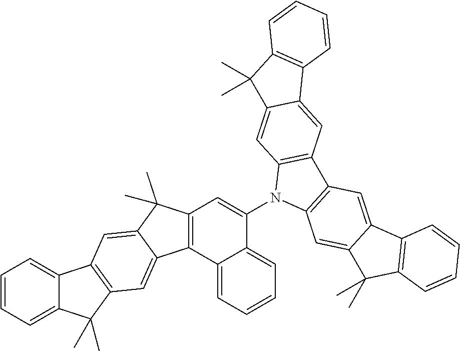 [SMILES: CC1(C)c2ccccc2-c2cc3c(cc21)-c1c(cc(-n2c4cc5c(cc4c4cc6c(cc42)C(C)(C)c2ccccc2-6)-c2ccccc2C5(C)C)c2ccccc12)C3(C)C] | (4) |
| 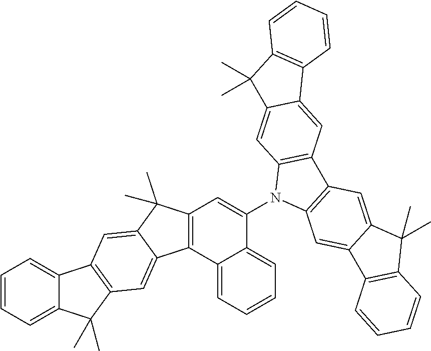 [SMILES: CC1(C)c2ccccc2-c2cc3c(cc21)-c1c(cc(-n2c4cc5c(cc4c4cc6c(cc42)C(C)(C)c2ccccc2-6)C(C)(C)c2ccccc2-5)c2ccccc12)C3(C)C] | (5) |
| 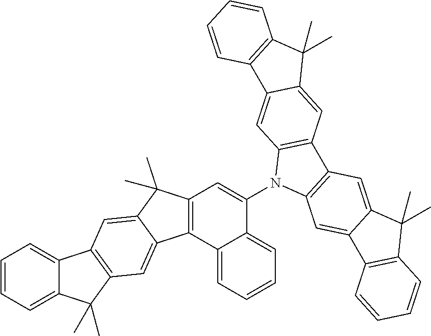 [SMILES: CC1(C)c2ccccc2-c2cc3c(cc21)-c1c(cc(-n2c4cc5c(cc4c4cc6c(cc42)-c2ccccc2C6(C)C)C(C)(C)c2ccccc2-5)c2ccccc12)C3(C)C] | (6) |
| 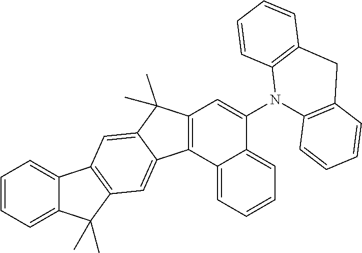 [SMILES: CC1(C)c2ccccc2-c2cc3c(cc21)-c1c(cc(N2c4ccccc4Cc4ccccc42)c2ccccc12)C3(C)C] | |
| 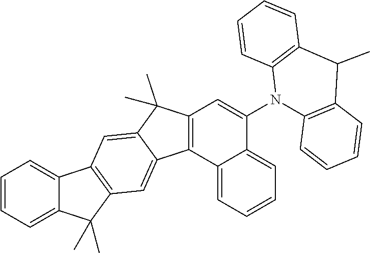 [SMILES: CC1c2ccccc2N(c2cc3c(c4ccccc24)-c2cc4c(cc2C3(C)C)-c2ccccc2C4(C)C)c2ccccc21] | |
| 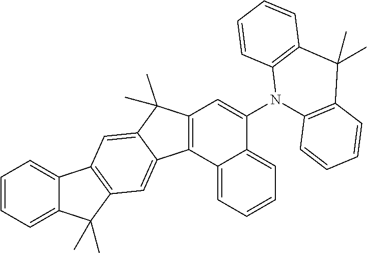 [SMILES: CC1(C)c2ccccc2-c2cc3c(cc21)-c1c(cc(N2c4ccccc4C(C)(C)c4ccccc42)c2ccccc12)C3(C)C] | (7) |
| 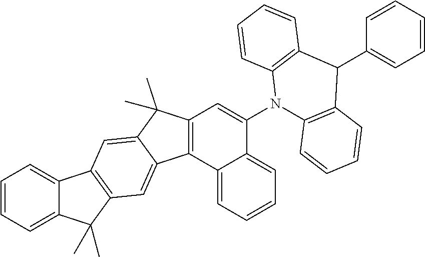 [SMILES: CC1(C)c2ccccc2-c2cc3c(cc21)-c1c(cc(N2c4ccccc4C(c4ccccc4)c4ccccc42)c2ccccc12)C3(C)C] | (8) |
| 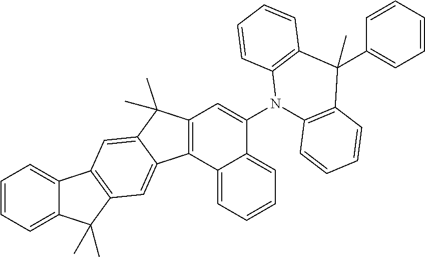 [SMILES: CC1(C)c2ccccc2-c2cc3c(cc21)-c1c(cc(N2c4ccccc4C(C)(c4ccccc4)c4ccccc42)c2ccccc12)C3(C)C] | (9) |
| 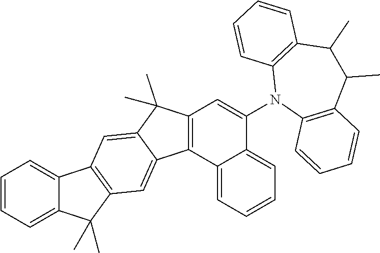 [SMILES: CC1c2ccccc2N(c2cc3c(c4ccccc24)-c2cc4c(cc2C3(C)C)-c2ccccc2C4(C)C)c2ccccc2C1C] | (10) |
| 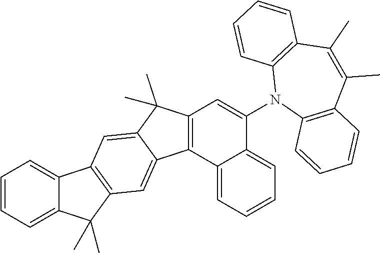 [SMILES: CC1=C(C)c2ccccc2N(c2cc3c(c4ccccc24)-c2cc4c(cc2C3(C)C)-c2ccccc2C4(C)C)c2ccccc21] | (11) |
| 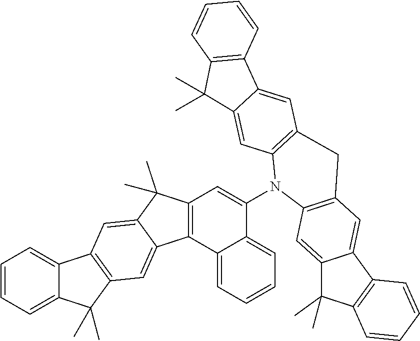 [SMILES: CC1(C)c2ccccc2-c2cc3c(cc21)-c1c(cc(N2c4cc5c(cc4Cc4cc6c(cc42)C(C)(C)c2ccccc2-6)-c2ccccc2C5(C)C)c2ccccc12)C3(C)C] | (12) |
| 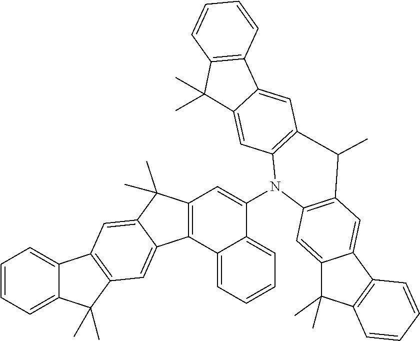 [SMILES: CC1c2cc3c(cc2N(c2cc4c(c5ccccc25)-c2cc5c(cc2C4(C)C)-c2ccccc2C5(C)C)c2cc4c(cc21)-c1ccccc1C4(C)C)C(C)(C)c1ccccc1-3] | (13) |
| 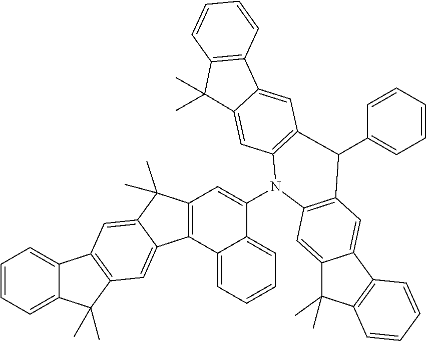 [SMILES: CC1(C)c2ccccc2-c2cc3c(cc21)-c1c(cc(N2c4cc5c(cc4C(c4ccccc4)c4cc6c(cc42)C(C)(C)c2ccccc2-6)-c2ccccc2C5(C)C)c2ccccc12)C3(C)C] | (14) |
| 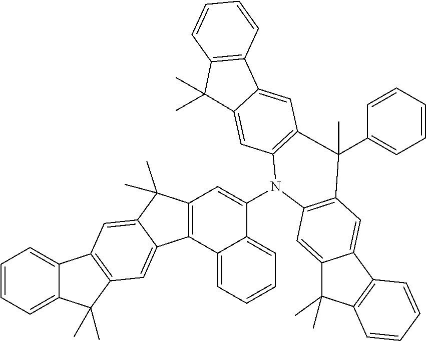 [SMILES: CC1(C)c2ccccc2-c2cc3c(cc21)-c1c(cc(N2c4cc5c(cc4C(C)(c4ccccc4)c4cc6c(cc42)C(C)(C)c2ccccc2-6)-c2ccccc2C5(C)C)c2ccccc12)C3(C)C] | (15) |
| 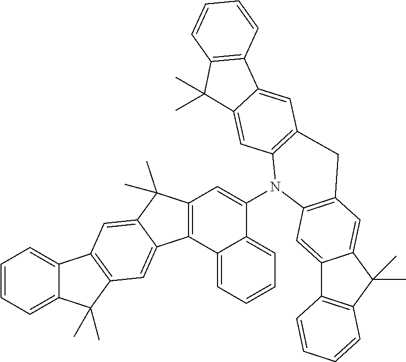 [SMILES: CC1(C)c2ccccc2-c2cc3c(cc21)Cc1cc2c(cc1N3c1cc3c(c4ccccc14)-c1cc4c(cc1C3(C)C)-c1ccccc1C4(C)C)C(C)(C)c1ccccc1-2] | (16) |
| 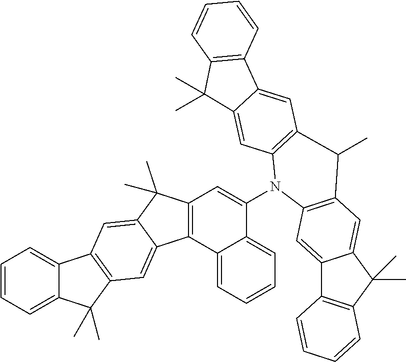 [SMILES: CC1c2cc3c(cc2N(c2cc4c(c5ccccc25)-c2cc5c(cc2C4(C)C)-c2ccccc2C5(C)C)c2cc4c(cc21)-c1ccccc1C4(C)C)-c1ccccc1C3(C)C] | (17) |
| 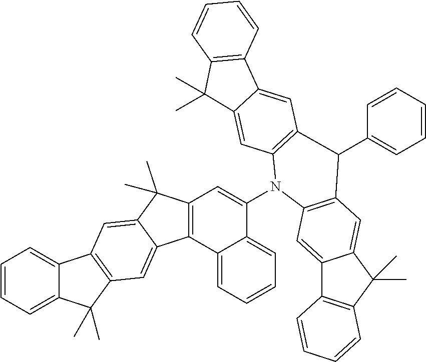 [SMILES: CC1(C)c2ccccc2-c2cc3c(cc21)-c1c(cc(N2c4cc5c(cc4C(c4ccccc4)c4cc6c(cc42)C(C)(C)c2ccccc2-6)C(C)(C)c2ccccc2-5)c2ccccc12)C3(C)C] | (18) |
| 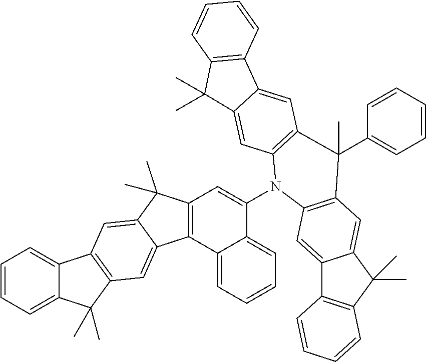 [SMILES: CC1(C)c2ccccc2-c2cc3c(cc21)-c1c(cc(N2c4cc5c(cc4C(C)(c4ccccc4)c4cc6c(cc42)-c2ccccc2C6(C)C)-c2ccccc2C5(C)C)c2ccccc12)C3(C)C] | (19) |
| 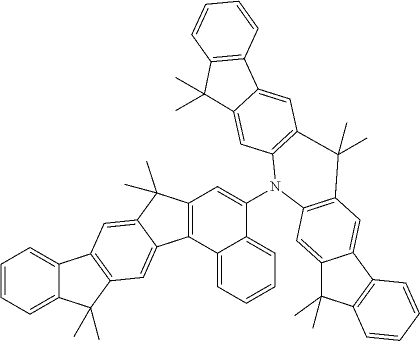 [SMILES: CC1(C)c2ccccc2-c2cc3c(cc21)-c1c(cc(N2c4cc5c(cc4C(C)(C)c4cc6c(cc42)C(C)(C)c2ccccc2-6)-c2ccccc2C5(C)C)c2ccccc12)C3(C)C] | (20) |
| 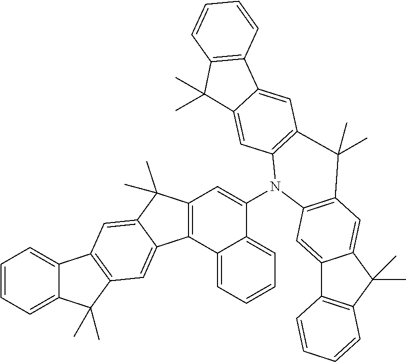 [SMILES: CC1(C)c2ccccc2-c2cc3c(cc21)-c1c(cc(N2c4cc5c(cc4C(C)(C)c4cc6c(cc42)-c2ccccc2C6(C)C)-c2ccccc2C5(C)C)c2ccccc12)C3(C)C] | (21) |
| 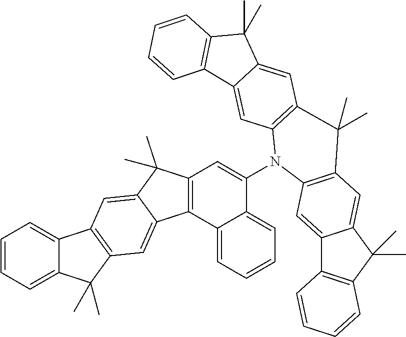 [SMILES: CC1(C)c2ccccc2-c2cc3c(cc21)-c1c(cc(N2c4cc5c(cc4C(C)(C)c4cc6c(cc42)-c2ccccc2C6(C)C)C(C)(C)c2ccccc2-5)c2ccccc12)C3(C)C] | (22) |
| 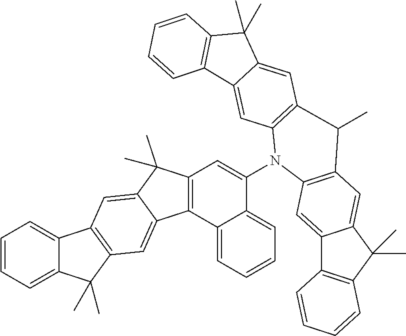 [SMILES: CC1c2cc3c(cc2N(c2cc4c(c5ccccc25)-c2cc5c(cc2C4(C)C)-c2ccccc2C5(C)C)c2cc4c(cc21)C(C)(C)c1ccccc1-4)-c1ccccc1C3(C)C] | (23) |
| 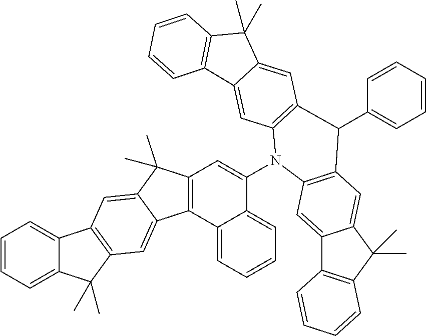 [SMILES: CC1(C)c2ccccc2-c2cc3c(cc21)-c1c(cc(N2c4cc5c(cc4C(c4ccccc4)c4cc6c(cc42)-c2ccccc2C6(C)C)C(C)(C)c2ccccc2-5)c2ccccc12)C3(C)C] | (24) |
| 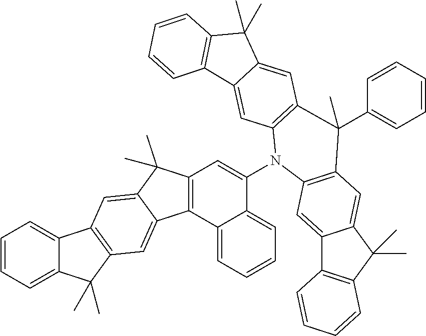 [SMILES: CC1(C)c2ccccc2-c2cc3c(cc21)-c1c(cc(N2c4cc5c(cc4C(C)(c4ccccc4)c4cc6c(cc42)-c2ccccc2C6(C)C)C(C)(C)c2ccccc2-5)c2ccccc12)C3(C)C] | (25) |
| 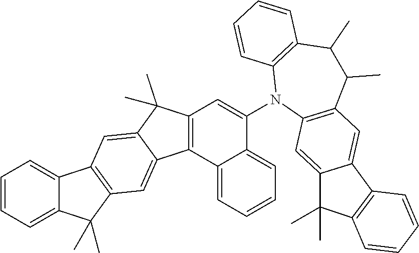 [SMILES: CC1c2ccccc2N(c2cc3c(c4ccccc24)-c2cc4c(cc2C3(C)C)-c2ccccc2C4(C)C)c2cc3c(cc2C1C)-c1ccccc1C3(C)C] | (26) |
| 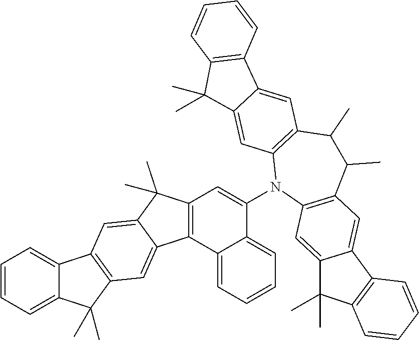 [SMILES: CC1c2cc3c(cc2N(c2cc4c(c5ccccc25)-c2cc5c(cc2C4(C)C)-c2ccccc2C5(C)C)c2cc4c(cc2C1C)-c1ccccc1C4(C)C)C(C)(C)c1ccccc1-3] | (27) |
| 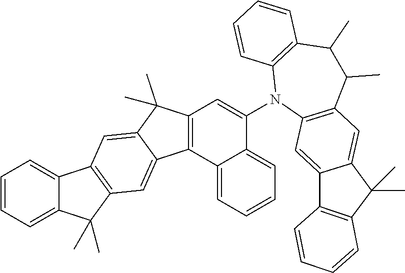 [SMILES: CC1c2ccccc2N(c2cc3c(c4ccccc24)-c2cc4c(cc2C3(C)C)-c2ccccc2C4(C)C)c2cc3c(cc2C1C)C(C)(C)c1ccccc1-3] | (28) |
| 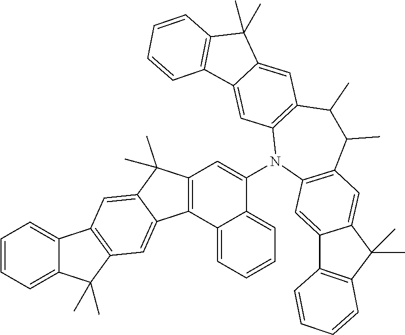 [SMILES: CC1c2cc3c(cc2N(c2cc4c(c5ccccc25)-c2cc5c(cc2C4(C)C)-c2ccccc2C5(C)C)c2cc4c(cc2C1C)C(C)(C)c1ccccc1-4)-c1ccccc1C3(C)C] | (29) |
| 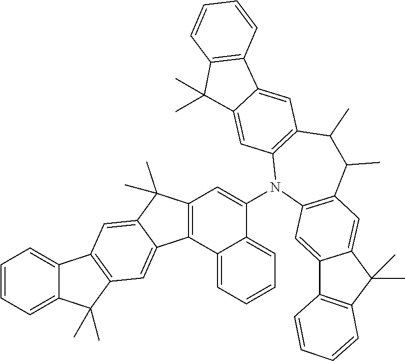 [SMILES: CC1c2cc3c(cc2N(c2cc4c(c5ccccc25)-c2cc5c(cc2C4(C)C)-c2ccccc2C5(C)C)c2cc4c(cc2C1C)-c1ccccc1C4(C)C)-c1ccccc1C3(C)C] | (30) |
| 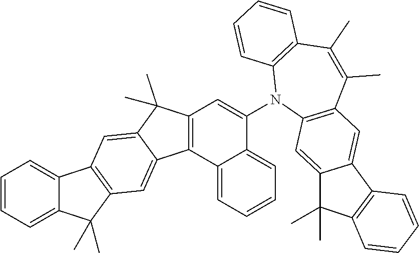 [SMILES: CC1=C(C)c2cc3c(cc2N(c2cc4c(c5ccccc25)-c2cc5c(cc2C4(C)C)-c2ccccc2C5(C)C)c2ccccc21)C(C)(C)c1ccccc1-3] | (31) |
| 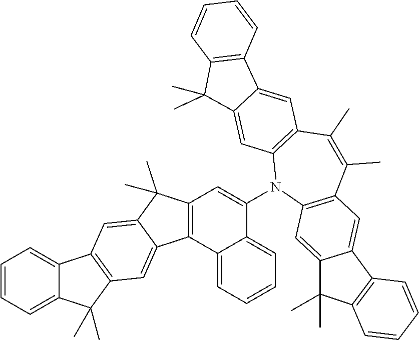 [SMILES: CC1=C(C)c2cc3c(cc2N(c2cc4c(c5ccccc25)-c2cc5c(cc2C4(C)C)-c2ccccc2C5(C)C)c2cc4c(cc21)-c1ccccc1C4(C)C)C(C)(C)c1ccccc1-3] | (32) |
| 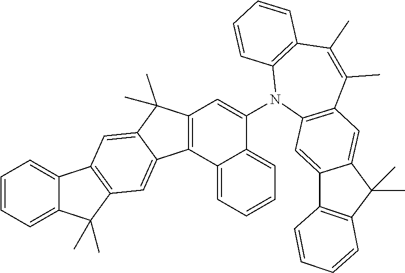 [SMILES: CC1=C(C)c2cc3c(cc2N(c2cc4c(c5ccccc25)-c2cc5c(cc2C4(C)C)-c2ccccc2C5(C)C)c2ccccc21)-c1ccccc1C3(C)C] | (33) |
| 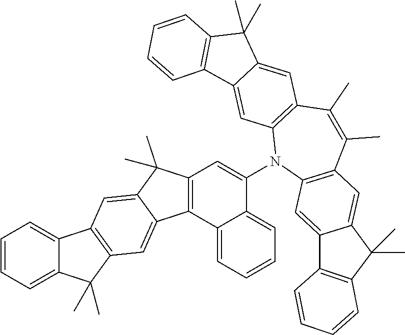 [SMILES: CC1=C(C)c2cc3c(cc2N(c2cc4c(c5ccccc25)-c2cc5c(cc2C4(C)C)-c2ccccc2C5(C)C)c2cc4c(cc21)C(C)(C)c1ccccc1-4)-c1ccccc1C3(C)C] | (34) |
| 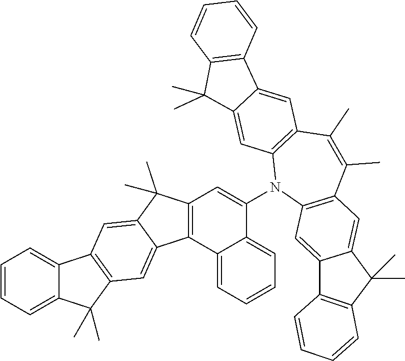 [SMILES: CC1=C(C)c2cc3c(cc2N(c2cc4c(c5ccccc25)-c2cc5c(cc2C4(C)C)-c2ccccc2C5(C)C)c2cc4c(cc21)C(C)(C)c1ccccc1-4)C(C)(C)c1ccccc1-3] | (35) |
| 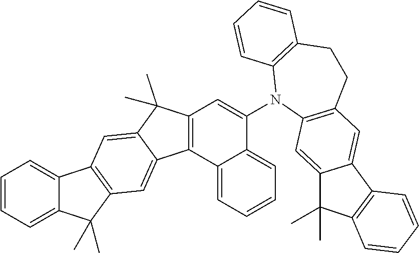 [SMILES: CC1(C)c2ccccc2-c2cc3c(cc21)-c1c(cc(N2c4ccccc4CCc4cc5c(cc42)C(C)(C)c2ccccc2-5)c2ccccc12)C3(C)C] | (36) |
| 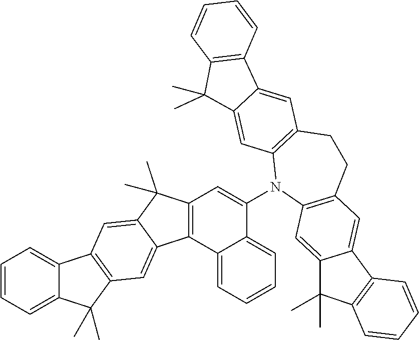 [SMILES: CC1(C)c2ccccc2-c2cc3c(cc21)-c1c(cc(N2c4cc5c(cc4CCc4cc6c(cc42)C(C)(C)c2ccccc2-6)-c2ccccc2C5(C)C)c2ccccc12)C3(C)C] | (37) |
| 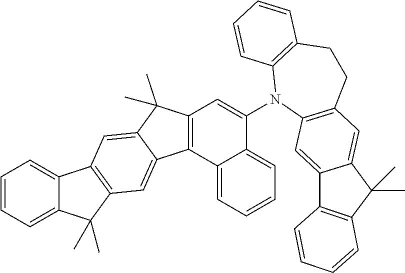 [SMILES: CC1(C)c2ccccc2-c2cc3c(cc21)CCc1ccccc1N3c1cc2c(c3ccccc13)-c1cc3c(cc1C2(C)C)-c1ccccc1C3(C)C] | (38) |
| 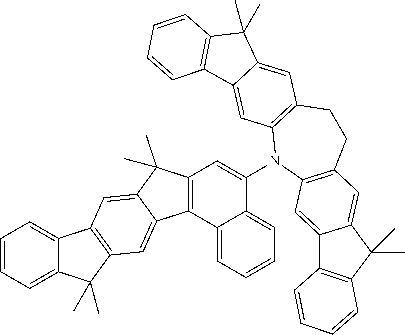 [SMILES: CC1(C)c2ccccc2-c2cc3c(cc21)CCc1cc2c(cc1N3c1cc3c(c4ccccc14)-c1cc4c(cc1C3(C)C)-c1ccccc1C4(C)C)-c1ccccc1C2(C)C] | (39) |
| 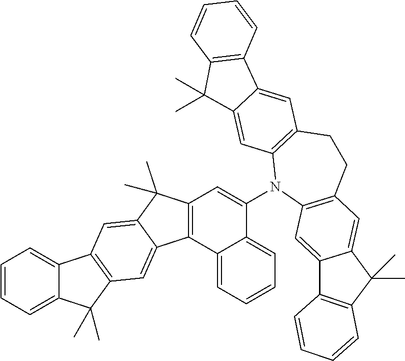 [SMILES: CC1(C)c2ccccc2-c2cc3c(cc21)CCc1cc2c(cc1N3c1cc3c(c4ccccc14)-c1cc4c(cc1C3(C)C)-c1ccccc1C4(C)C)C(C)(C)c1ccccc1-2] | (40) |
| 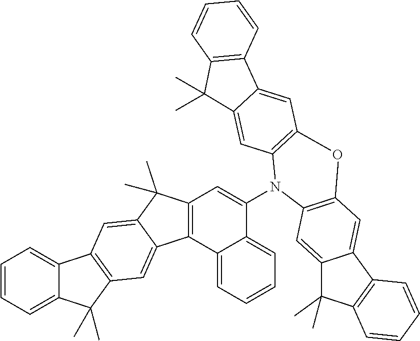 [SMILES: CC1(C)c2ccccc2-c2cc3c(cc21)-c1c(cc(N2c4cc5c(cc4Oc4cc6c(cc42)C(C)(C)c2ccccc2-6)-c2ccccc2C5(C)C)c2ccccc12)C3(C)C] | (41) |
| 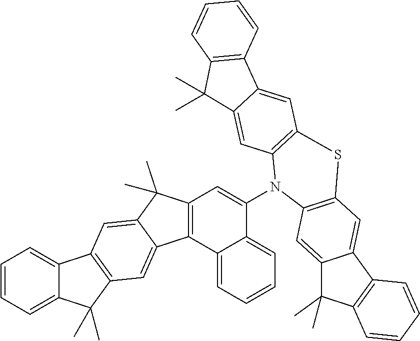 [SMILES: CC1(C)c2ccccc2-c2cc3c(cc21)-c1c(cc(N2c4cc5c(cc4Sc4cc6c(cc42)C(C)(C)c2ccccc2-6)-c2ccccc2C5(C)C)c2ccccc12)C3(C)C] | (42) |
| 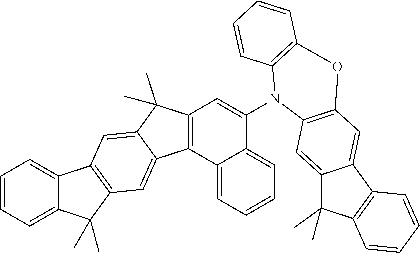 [SMILES: CC1(C)c2ccccc2-c2cc3c(cc21)-c1c(cc(N2c4ccccc4Oc4cc5c(cc42)C(C)(C)c2ccccc2-5)c2ccccc12)C3(C)C] | (43) |
| 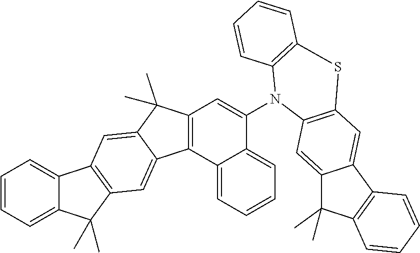 [SMILES: CC1(C)c2ccccc2-c2cc3c(cc21)-c1c(cc(N2c4ccccc4Sc4cc5c(cc42)C(C)(C)c2ccccc2-5)c2ccccc12)C3(C)C] | (44) |
| 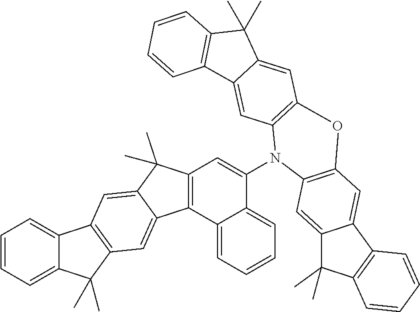 [SMILES: CC1(C)c2ccccc2-c2cc3c(cc21)Oc1cc2c(cc1N3c1cc3c(c4ccccc14)-c1cc4c(cc1C3(C)C)-c1ccccc1C4(C)C)C(C)(C)c1ccccc1-2] | (45) |
| 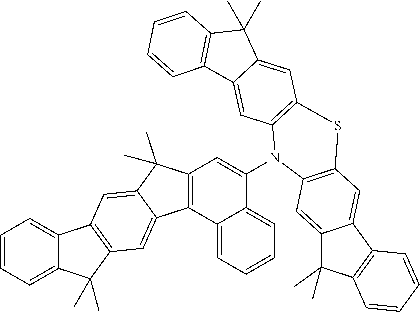 [SMILES: CC1(C)c2ccccc2-c2cc3c(cc21)Sc1cc2c(cc1N3c1cc3c(c4ccccc14)-c1cc4c(cc1C3(C)C)-c1ccccc1C4(C)C)C(C)(C)c1ccccc1-2] | (46) |
| 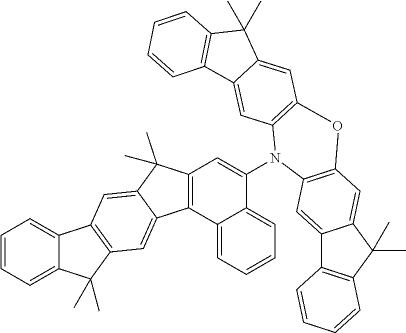 [SMILES: CC1(C)c2ccccc2-c2cc3c(cc21)Oc1cc2c(cc1N3c1cc3c(c4ccccc14)-c1cc4c(cc1C3(C)C)-c1ccccc1C4(C)C)-c1ccccc1C2(C)C] | (47) |
| 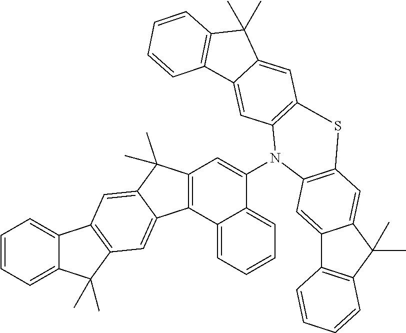 [SMILES: CC1(C)c2ccccc2-c2cc3c(cc21)Sc1cc2c(cc1N3c1cc3c(c4ccccc14)-c1cc4c(cc1C3(C)C)-c1ccccc1C4(C)C)-c1ccccc1C2(C)C] | (48) |

The compounds of the formula (I) can be prepared by means of standard reactions in organic synthetic chemistry, especially by acid-catalyzed ring-closing reactions of tertiary alcohols, by Suzuki coupling reactions and by Buchwald coupling reactions.

A suitable synthesis method for preparation of a compound of the formula (I) is shown below (scheme 1). Further details in this regard can be found in the working examples.

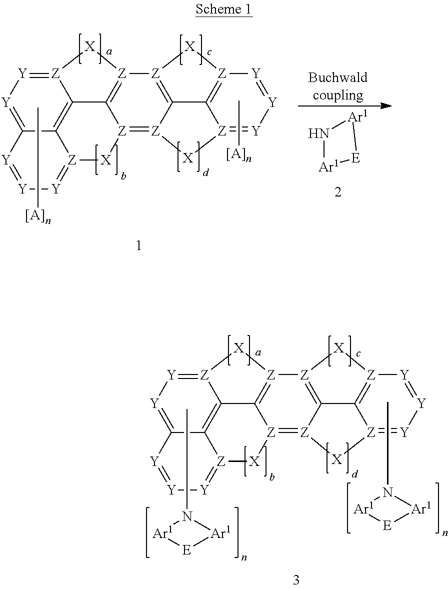

where the variables that occur are as defined above, and in addition:
A=reactive group, preferably halogen, Sn-organyl, Si-organyl, mesylate or triflate, more preferably Cl, Br or I.
n=0, 1 or 2, preferably 0 or 1, where the sum total of all n is preferably 1.

In the process shown, a monobenzoindenofluorene derivative 1 containing one or more reactive groups is reacted with a secondary amine 2 in a Buchwald reaction. This affords the target compound 3 of formula (I). Said reactant 1 can be prepared as described in WO 2008/006449 A1. The reactants 2 are either known from the literature, such as the dimethylindenocarbazole, the synthesis of which is disclosed in WO 2011/050888 A1 at pages 73-75, or can be prepared by processes known to those skilled in the art.

The present invention thus further provides a process for preparing a compound of the formula (I), characterized in that a monobenzoindenofluorene derivative comprising one or more reactive groups is reacted with an amine in a transition metal-catalyzed coupling reaction.

Preferably, the transition metal-catalyzed coupling reaction is a Buchwald coupling.

The reactive groups are preferably selected from Cl, Br, I, Sn-organyls, Si-organyls, mesylates and triflates. Preferably, the monobenzoindenofluorene derivative comprises exactly one reactive group.

The above-described compounds of the invention, especially compounds substituted by reactive leaving groups, such as bromine, iodine, chlorine, boronic acid or boronic ester, may find use as monomers for production of corresponding oligomers, dendrimers or polymers. Suitable reactive leaving groups are, for example, bromine, iodine, chlorine, boronic acids, boronic esters, amines, alkenyl or alkynyl groups having a terminal C—C double bond or C—C triple bond, oxiranes, oxetanes, groups which enter into a cycloaddition, for example a 1,3-dipolar cycloaddition, for example dienes or azides, carboxylic acid derivatives, alcohols and silanes.

The invention therefore further provides oligomers, polymers or dendrimers containing one or more compounds of formula (I), wherein the bond(s) to the polymer, oligomer or dendrimer may be localized at any desired R¹- or R²-substituted positions in formula (I). According to the linkage of the compound of formula (I), the compound is part of a side chain of the oligomer or polymer or part of the main chain. An oligomer in the context of this invention is understood to mean a compound formed from at least three monomer units. A polymer in the context of the invention is understood to mean a compound formed from at least ten monomer units. The polymers, oligomers or dendrimers of the invention may be conjugated, partly conjugated or nonconjugated. The oligomers or polymers of the invention may be linear, branched or dendritic. In the structures having linear linkage, the units of formula (I) may be joined directly to one another, or they may be joined to one another via a bivalent group, for example via a substituted or unsubstituted alkylene group, via a heteroatom or via a bivalent aromatic or heteroaromatic group. In branched and dendritic structures, it is possible, for example, for three or more units of formula (I) to be joined via a trivalent or higher-valency group, for example via a trivalent or higher-valency aromatic or heteroaromatic group, to give a branched or dendritic oligomer or polymer.

For the repeat units of formula (I) in oligomers, dendrimers and polymers, the same preferences apply as described above for compounds of formula (I).

For preparation of the oligomers or polymers, the monomers of the invention are homopolymerized or copolymerized with further monomers. Suitable and preferred comonomers are selected from fluorenes (for example according to EP 842208 or WO 00/22026), spirobifluorenes (for example according to EP 707020, EP 894107 or WO 06/061181), paraphenylenes (for example according to WO 1992/18552), carbazoles (for example according to WO 04/070772 or WO 2004/113468), thiophenes (for example according to EP 1028136), dihydrophenanthrenes (for example according to WO 2005/014689 or WO 2007/006383), cis- and trans-indenofluorenes (for example according to WO 2004/041901 or WO 2004/113412), ketones (for example according to WO 2005/040302), phenanthrenes (for example according to WO 2005/104264 or WO 2007/017066) or else a plurality of these units. The polymers, oligomers and dendrimers typically contain still further units, for example emitting (fluorescent or phosphorescent) units, for example vinyltriarylamines (for example according to WO 2007/068325) or phosphorescent metal complexes (for example according to WO 2006/003000), and/or charge transport units, especially those based on triarylamines.

The polymers and oligomers of the invention are generally prepared by polymerization of one or more monomer types, of which at least one monomer leads to repeat units of the formula (I) in the polymer. Suitable polymerization reactions are known to those skilled in the art and are described in the literature. Particularly suitable and preferred polymerization reactions which lead to C—C and C—N couplings are as follows:
(A) SUZUKI polymerization;
(B) YAMAMOTO polymerization;
(C) STILLE polymerization; and
(D) HARTWIG-BUCHWALD polymerization.

How the polymerization can be conducted by these methods and how the polymers can then be separated from the reaction medium and purified is known to those skilled in the art and is described in detail in the literature, for example in WO 2003/048225, WO 2004/037887 and WO 2004/037887.

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclohexanol, cyclohexanone, cyclohexylbenzene, decalin, dodecylbenzene, ethyl benzoate, indane, methyl benzoate, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane or mixtures of these solvents.

The invention therefore further provides a formulation, especially a solution, dispersion or emulsion, comprising at least one compound of formula (I) or at least one polymer, oligomer or dendrimer containing at least one unit of formula (I) and at least one solvent, preferably an organic solvent. The way in which such solutions can be prepared is known to those skilled in the art and is described, for example, in WO 2002/072714, WO 2003/019694 and the literature cited therein.

The compounds of formula (I) are suitable for use in electronic devices, especially in organic electroluminescent devices (OLEDs). Depending on the substitution, the compounds are used in different functions and layers.

The compound of the formula (I) can be used in any function in the organic electroluminescent device, for example as hole-transporting material, as matrix material, as emitting material, or as electron-transporting material.

The invention therefore further provides for the use of a compound of formula (I) in an electronic device. This electronic device is preferably selected from the group consisting of organic integrated circuits (OICs), organic field-effect transistors (OFETs), organic thin-film transistors (OTFTs), organic light-emitting transistors (OLETs), organic solar cells (OSCs), organic optical detectors, organic photoreceptors, organic field-quench devices (OFQDs), organic light-emitting electrochemical cells (OLECs), organic laser diodes (O-lasers) and more preferably organic electroluminescent devices (OLEDs).

The invention further provides an electronic device comprising at least one compound of the formula (I). The electronic device is preferably selected from the above-specified devices. Particular preference is given to an organic electroluminescent device comprising anode, cathode and at least one emitting layer, characterized in that at least one organic layer comprises at least one compound of formula (I).

Apart from the cathode, anode and emitting layer, the organic electroluminescent device may also comprise further layers. These are selected, for example, from in each case one or more hole injection layers, hole transport layers, hole blocker layers, electron transport layers, electron injection layers, electron blocker layers, exciton blocker layers, interlayers, charge generation layers (IDMC 2003, Taiwan; Session 21 OLED (5), T. Matsumoto, T. Nakada, J. Endo, K. Mori, N. Kawamura, A. Yokoi, J. Kido, Multiphoton Organic EL Device Having Charge Generation Layer) and/or organic or inorganic p/n junctions.

The sequence of layers in the organic electroluminescent device is preferably as follows: anode-hole injection layer-hole transport layer-emitting layer-electron transport layer-electron injection layer-cathode. Not all the layers mentioned need be present here, and it is additionally possible for further layers to be present, for example an electron blocker layer adjoining the emitting layer on the anode side, or a hole blocker layer adjoining the emitting layer on the cathode side.

The organic electroluminescent device of the invention preferably comprises two or more layers having hole-transporting function between anode and emitting layer.

In a hole-transporting layer, the hole transport material may be used as a pure material, for example in a proportion of 100%, or it can be used in combination with one or more further compounds. In a preferred embodiment, at least one hole transport layer of the OLED, in addition to the hole transport material, comprises one or more p-dopants. p-Dopants used according to the present invention are preferably those organic electron acceptor compounds capable of oxidizing one or more of the other compounds in the mixture.

Particularly preferred embodiments of p-dopants are the compounds disclosed in WO 2011/073149, EP 1968131, EP 2276085, EP 2213662, EP 1722602, EP 2045848, DE 102007031220, U.S. Pat. No. 8,044,390, U.S. Pat. No. 8,057,712, WO 2009/003455, WO 2010/094378, WO 2011/120709, US 2010/0096600 and WO 2012/095143.

The organic electroluminescent device of the invention may contain two or more emitting layers. More preferably, these emission layers in this case have several emission maxima between 380 nm and 750 nm overall, such that the overall result is white emission; in other words, various emitting compounds which may fluoresce or phosphoresce and which emit blue, green, yellow, orange or red light are used in the emitting layers. Especially preferred are three-layer systems, i.e. systems having three emitting layers, where preferably at least one of these layers comprises at least one compound of formula (I) and where the three layers show blue, green, yellow, orange or red emission (for the basic construction see, for example, WO 2005/011013). It should be noted that, for the production of white light, rather than a plurality of color-emitting emitter compounds, an emitter compound used individually which emits over a broad wavelength range may also be suitable. Alternatively and/or additionally, the compounds of the invention in such an organic electroluminescent device may also be present in the hole transport layer or in another layer. The various emitting layers may directly adjoin one another, or they may be separated from one another by non-emitting layers. In a preferred embodiment of the invention, a white-emitting OLED is what is called a tandem OLED, meaning that two or more complete OLED layer sequences are present in the OLED, the OLED layer sequences each comprising hole transport layer, emitting layer and electron transport layer, each of which are separated from one another by a charge generation layer.

It is preferable when the compound of formula (I) is used in an emitting layer. The compound of formula (I) is especially suitable for use as an emitting compound, particularly as a blue-emitting compound or as a compound that emits in the near UV.

When the compound of the invention is used as emitting compound in an emitting layer, it is preferably used in combination with one or more matrix materials. A matrix material is understood here to mean a material which is present in the emitting layer, preferably as main component, and which does not emit light in the operation of the device.

The proportion of the emitting compound in the mixture of the emitting layer is between 0.1% and 50.0%, preferably between 0.5% and 20.0%, more preferably between 1.0% and 10.0%. Correspondingly, the proportion of the matrix material(s) is between 50.0% and 99.9%, preferably between 80.0% and 99.5%, more preferably between 90.0% and 99.0%.

The FIGURES for the proportions in % are understood in the context of the present application to mean % by volume when the compounds are applied from the gas phase, and to mean % by weight when the compounds are applied from solution.

Detailed hereinafter are generally preferred material classes for use as corresponding functional materials in the organic electroluminescent devices of the invention.

Suitable phosphorescent emitting compounds are especially compounds which, when suitably excited, emit light, preferably in the visible region, and also contain at least one atom of atomic number greater than 20, preferably greater than 38, and less than 84, more preferably greater than 56 and less than 80. Preference is given to using, as phosphorescent emitting compounds, compounds containing copper, molybdenum, tungsten, rhenium, ruthenium, osmium, rhodium, iridium, palladium, platinum, silver, gold or europium, especially compounds containing iridium, platinum or copper.

In the context of the present invention, all luminescent iridium, platinum or copper complexes are considered to be phosphorescent compounds.

Examples of the above-described phosphorescent emitting compounds can be found in applications WO 2000/70655, WO 2001/41512, WO 2002/02714, WO 2002/15645, EP 1191613, EP 1191612, EP 1191614, WO 2005/033244, WO 2005/019373 and US 2005/0258742. In general, all phosphorescent complexes as used for phosphorescent OLEDs according to the prior art and as known to those skilled in the art in the field of organic electroluminescent devices are suitable for use in the devices of the invention. It is also possible for the person skilled in the art, without exercising inventive skill, to use further phosphorescent complexes in combination with the compounds of the invention in OLEDs.

Preferred fluorescent emitters are, aside from the compounds of the invention, selected from the class of the arylamines. An arylamine in the context of this invention is understood to mean a compound containing three substituted or unsubstituted aromatic or heteroaromatic ring systems bonded directly to the nitrogen. Preferably, at least one of these aromatic or heteroaromatic ring systems is a fused ring system, more preferably having at least 14 aromatic ring atoms. Preferred examples of these are aromatic anthraceneamines, aromatic anthracenediamines, aromatic pyreneamines, aromatic pyrenediamines, aromatic chryseneamines or aromatic chrysenediamines. An aromatic anthraceneamine is understood to mean a compound in which a diarylamino group is bonded directly to an anthracene group, preferably in the 9 position. An aromatic anthracenediamine is understood to mean a compound in which two diarylamino groups are bonded directly to an anthracene group, preferably in the 9,10 positions. Aromatic pyreneamines, pyrenediamines, chryseneamines and chrysenediamines are defined analogously, where the diarylamino groups are bonded to the pyrene preferably in the 1 position or 1,6 positions. Further preferred emitters are indenofluoreneamines or -diamines, for example according to WO 2006/108497 or WO 2006/122630, benzoindenofluoreneamines or -diamines, for example according to WO 2008/006449, and dibenzoindenofluoreneamines or -diamines, for example according to WO 2007/140847, and the indenofluorene derivatives having fused aryl groups disclosed in WO 2010/012328. Likewise preferred are the pyrenearylamines disclosed in WO 2012/048780 and WO 2013/185871. Likewise preferred are the benzoindenofluoreneamines disclosed in WO 2014/037077, the benzofluoreneamines disclosed in WO 2014/106522 and the extended indenofluorenes disclosed in WO 2014/111269.

Preferred matrix materials for use in combination with fluorescent emitting compounds are selected from the classes of the oligoarylenes (e.g. 2,2′,7,7′-tetraphenylspirobifluorene according to EP 676461 or dinaphthylanthracene), especially of the oligoarylenes containing fused aromatic groups, the oligoarylenevinylenes (e.g. DPVBi or spiro-DPVBi according to EP 676461), the polypodal metal complexes (for example according to WO 2004/081017), the hole-conducting compounds (for example according to WO 2004/058911), the electron-conducting compounds, especially ketones, phosphine oxides, sulfoxides, etc. (for example according to WO 2005/084081 and WO 2005/084082), the atropisomers (for example according to WO 2006/048268), the boronic acid derivatives (for example according to WO 2006/117052) or the benzanthracenes (for example according to WO 2008/145239). Particularly preferred matrix materials are selected from the classes of the oligoarylenes comprising naphthalene, anthracene, benzanthracene and/or pyrene or atropisomers of these compounds, the oligoarylenevinylenes, the ketones, the phosphine oxides and the sulfoxides. Very particularly preferred matrix materials are selected from the classes of the oligoarylenes comprising anthracene, benzanthracene, benzophenanthrene and/or pyrene or atropisomers of these compounds. An oligoarylene in the context of this invention shall be understood to mean a compound in which at least three aryl or arylene groups are bonded to one another.

Particularly preferred matrix materials for use in combination with the compound of the formula (I) in the emitting layer are depicted in the following table:

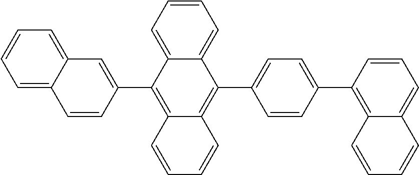 [SMILES: c1ccc2cc(-c3c4ccccc4c(-c4ccc(-c5cccc6ccccc56)cc4)c4ccccc34)ccc2c1]

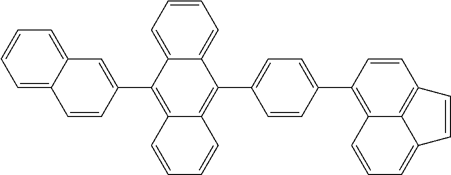 [SMILES: C1=Cc2ccc(-c3ccc(-c4c5ccccc5c(-c5ccc6ccccc6c5)c5ccccc45)cc3)c3cccc1c23]

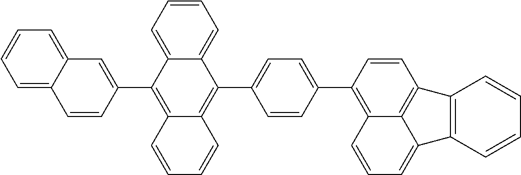 [SMILES: c1ccc2c(c1)-c1cccc3c(-c4ccc(-c5c6ccccc6c(-c6ccc7ccccc7c6)c6ccccc56)cc4)ccc-2c13]

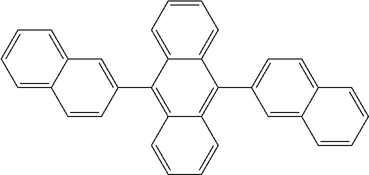 [SMILES: c1ccc2cc(-c3c4ccccc4c(-c4ccc5ccccc5c4)c4ccccc34)ccc2c1]

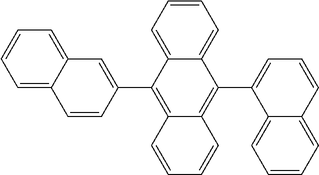 [SMILES: c1ccc2cc(-c3c4ccccc4c(-c4cccc5ccccc45)c4ccccc34)ccc2c1]

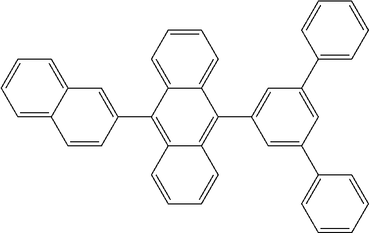 [SMILES: c1ccc(-c2cc(-c3ccccc3)cc(-c3c4ccccc4c(-c4ccc5ccccc5c4)c4ccccc34)c2)cc1]

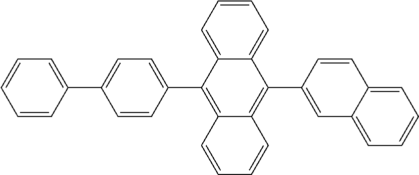 [SMILES: c1ccc(-c2ccc(-c3c4ccccc4c(-c4ccc5ccccc5c4)c4ccccc34)cc2)cc1]

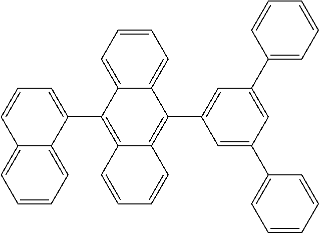 [SMILES: c1ccc(-c2cc(-c3ccccc3)cc(-c3c4ccccc4c(-c4cccc5ccccc45)c4ccccc34)c2)cc1]

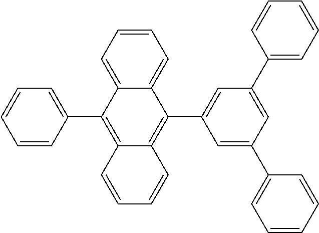 [SMILES: c1ccc(-c2cc(-c3ccccc3)cc(-c3c4ccccc4c(-c4ccccc4)c4ccccc34)c2)cc1]

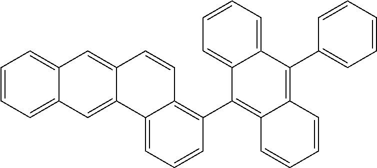 [SMILES: c1ccc(-c2c3ccccc3c(-c3cccc4c3ccc3cc5ccccc5cc34)c3ccccc23)cc1]

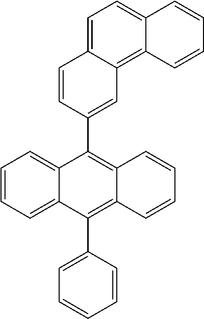 [SMILES: c1ccc(-c2c3ccccc3c(-c3ccc4ccc5ccccc5c4c3)c3ccccc23)cc1]

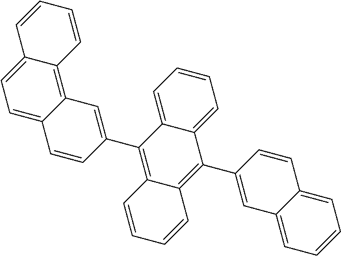 [SMILES: c1ccc2cc(-c3c4ccccc4c(-c4ccc5ccc6ccccc6c5c4)c4ccccc34)ccc2c1]

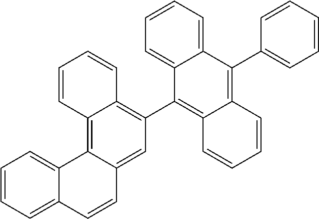 [SMILES: c1ccc(-c2c3ccccc3c(-c3cc4ccc5ccccc5c4c4ccccc34)c3ccccc23)cc1]

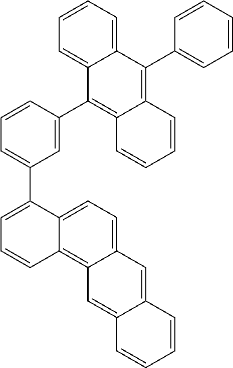 [SMILES: c1ccc(-c2c3ccccc3c(-c3cccc(-c4cccc5c4ccc4cc6ccccc6cc45)c3)c3ccccc23)cc1]

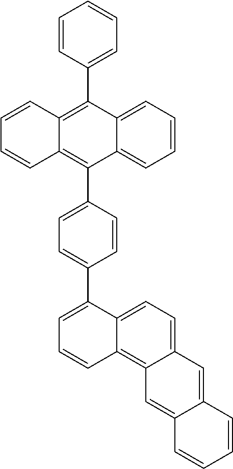 [SMILES: c1ccc(-c2c3ccccc3c(-c3ccc(-c4cccc5c4ccc4cc6ccccc6cc45)cc3)c3ccccc23)cc1]

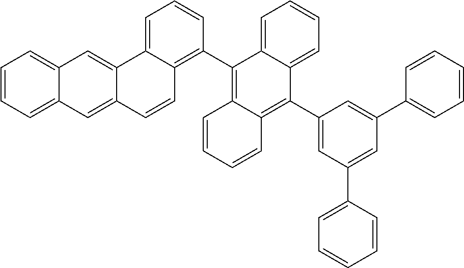 [SMILES: c1ccc(-c2cc(-c3ccccc3)cc(-c3c4ccccc4c(-c4cccc5c4ccc4cc6ccccc6cc45)c4ccccc34)c2)cc1]

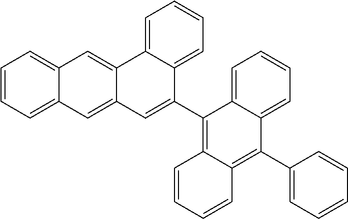 [SMILES: c1ccc(-c2c3ccccc3c(-c3cc4cc5ccccc5cc4c4ccccc34)c3ccccc23)cc1]

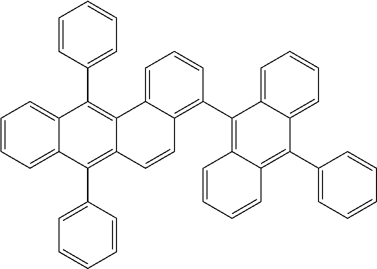 [SMILES: c1ccc(-c2c3ccccc3c(-c3cccc4c3ccc3c(-c5ccccc5)c5ccccc5c(-c5ccccc5)c34)c3ccccc23)cc1]

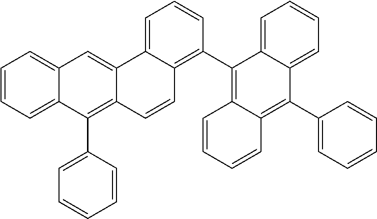 [SMILES: c1ccc(-c2c3ccccc3c(-c3cccc4c3ccc3c(-c5ccccc5)c5ccccc5cc34)c3ccccc23)cc1]

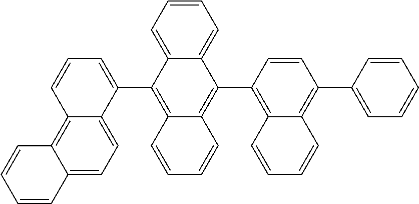 [SMILES: c1ccc(-c2ccc(-c3c4ccccc4c(-c4cccc5c4ccc4ccccc45)c4ccccc34)c3ccccc23)cc1]

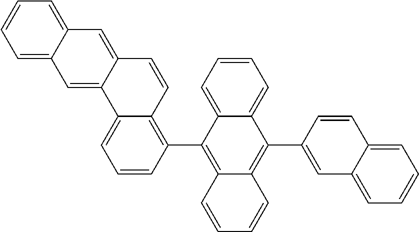 [SMILES: c1ccc2cc(-c3c4ccccc4c(-c4cccc5c4ccc4cc6ccccc6cc45)c4ccccc34)ccc2c1]

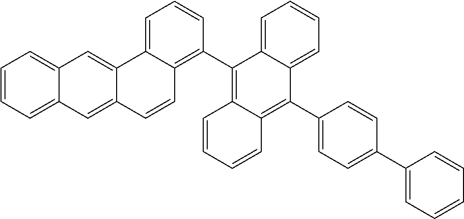 [SMILES: c1ccc(-c2ccc(-c3c4ccccc4c(-c4cccc5c4ccc4cc6ccccc6cc45)c4ccccc34)cc2)cc1]

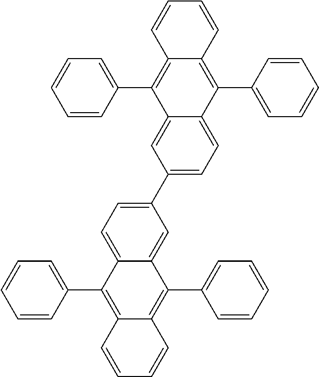 [SMILES: c1ccc(-c2c3ccccc3c(-c3ccccc3)c3cc(-c4ccc5c(-c6ccccc6)c6ccccc6c(-c6ccccc6)c5c4)ccc23)cc1]

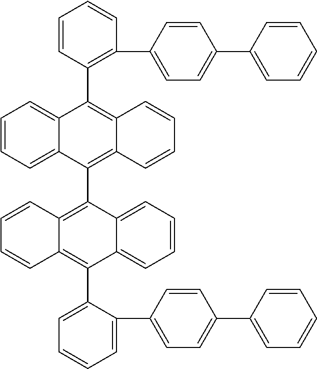 [SMILES: c1ccc(-c2ccc(-c3ccccc3-c3c4ccccc4c(-c4c5ccccc5c(-c5ccccc5-c5ccc(-c6ccccc6)cc5)c5ccccc45)c4ccccc34)cc2)cc1]

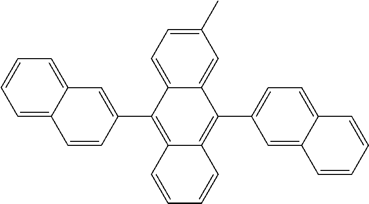 [SMILES: Cc1ccc2c(-c3ccc4ccccc4c3)c3ccccc3c(-c3ccc4ccccc4c3)c2c1]

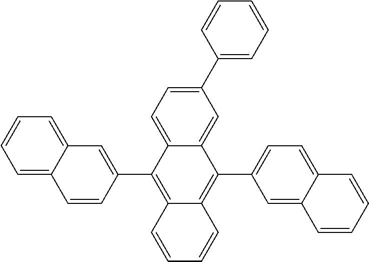 [SMILES: c1ccc(-c2ccc3c(-c4ccc5ccccc5c4)c4ccccc4c(-c4ccc5ccccc5c4)c3c2)cc1]

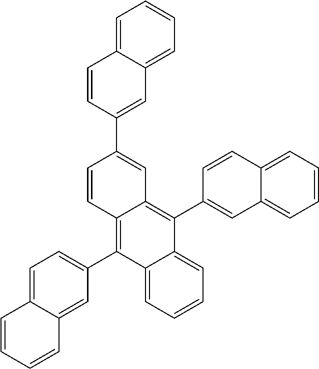 [SMILES: c1ccc2cc(-c3ccc4c(-c5ccc6ccccc6c5)c5ccccc5c(-c5ccc6ccccc6c5)c4c3)ccc2c1]

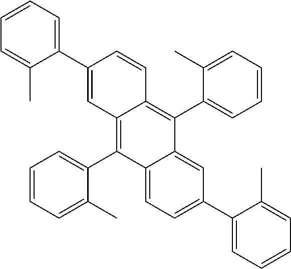 [SMILES: Cc1ccccc1-c1ccc2c(-c3ccccc3C)c3cc(-c4ccccc4C)ccc3c(-c3ccccc3C)c2c1]

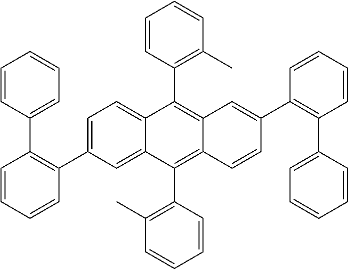 [SMILES: Cc1ccccc1-c1c2ccc(-c3ccccc3-c3ccccc3)cc2c(-c2ccccc2C)c2ccc(-c3ccccc3-c3ccccc3)cc12]

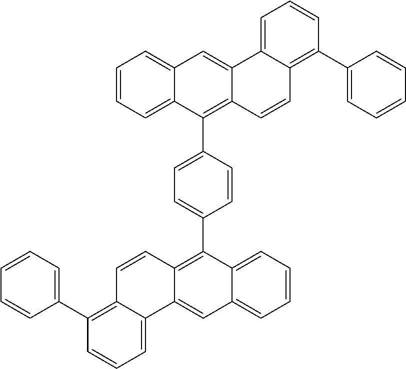 [SMILES: c1ccc(-c2cccc3c2ccc2c(-c4ccc(-c5c6ccccc6cc6c5ccc5c(-c7ccccc7)cccc56)cc4)c4ccccc4cc23)cc1]

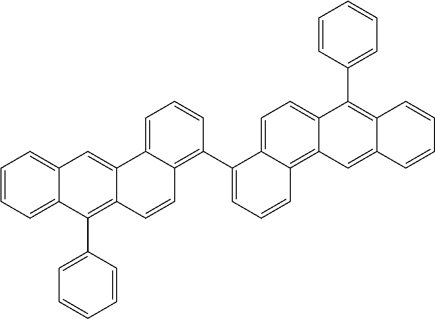 [SMILES: c1ccc(-c2c3ccccc3cc3c2ccc2c(-c4cccc5c4ccc4c(-c6ccccc6)c6ccccc6cc45)cccc23)cc1]

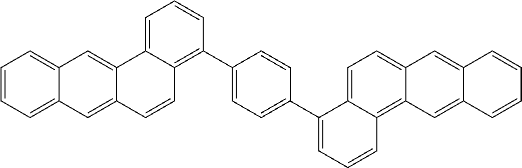 [SMILES: c1ccc2cc3c(ccc4c(-c5ccc(-c6cccc7c6ccc6cc8ccccc8cc67)cc5)cccc43)cc2c1]

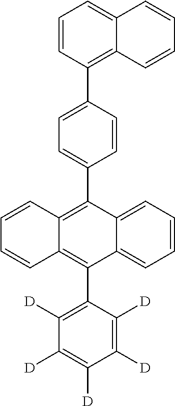 [SMILES: [2H]c1c([2H])c([2H])c(-c2c3ccccc3c(-c3ccc(-c4cccc5ccccc45)cc3)c3ccccc23)c([2H])c1[2H]]

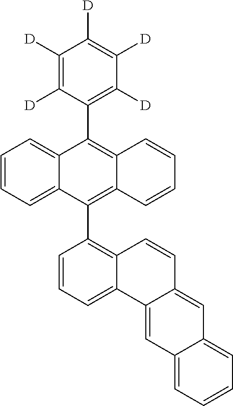 [SMILES: [2H]c1c([2H])c([2H])c(-c2c3ccccc3c(-c3cccc4c3ccc3cc5ccccc5cc34)c3ccccc23)c([2H])c1[2H]]

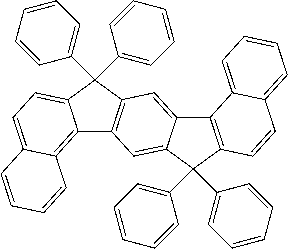 [SMILES: c1ccc(C2(c3ccccc3)c3cc4c(cc3-c3c2ccc2ccccc32)C(c2ccccc2)(c2ccccc2)c2ccc3ccccc3c2-4)cc1]

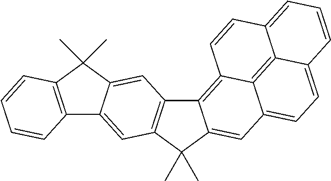 [SMILES: CC1(C)c2ccccc2-c2cc3c(cc21)-c1c(cc2ccc4cccc5ccc1c2c45)C3(C)C]

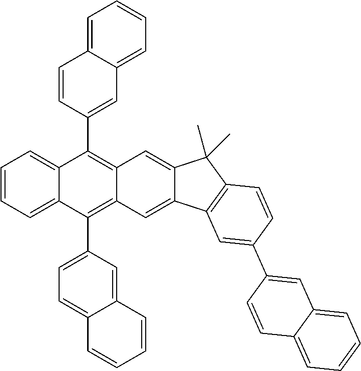 [SMILES: CC1(C)c2ccc(-c3ccc4ccccc4c3)cc2-c2cc3c(-c4ccc5ccccc5c4)c4ccccc4c(-c4ccc5ccccc5c4)c3cc21]

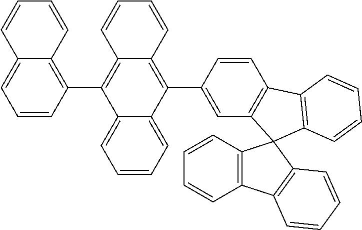 [SMILES: c1ccc2c(c1)-c1ccccc1C21c2ccccc2-c2ccc(-c3c4ccccc4c(-c4cccc5ccccc45)c4ccccc34)cc21]

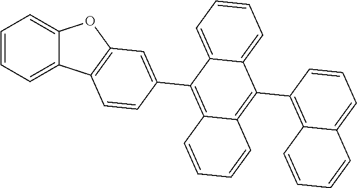 [SMILES: c1ccc2c(-c3c4ccccc4c(-c4ccc5c(c4)oc4ccccc45)c4ccccc34)cccc2c1]

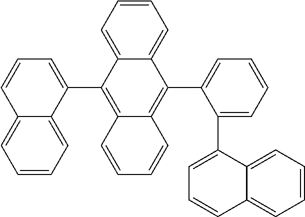 [SMILES: c1ccc(-c2c3ccccc3c(-c3cccc4ccccc34)c3ccccc23)c(-c2cccc3ccccc23)c1]

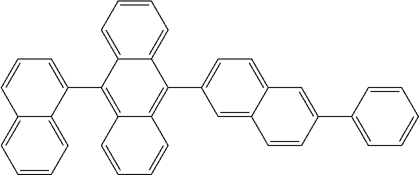 [SMILES: c1ccc(-c2ccc3cc(-c4c5ccccc5c(-c5cccc6ccccc56)c5ccccc45)ccc3c2)cc1]

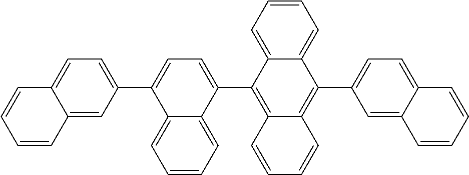 [SMILES: c1ccc2cc(-c3ccc(-c4c5ccccc5c(-c5ccc6ccccc6c5)c5ccccc45)c4ccccc34)ccc2c1]

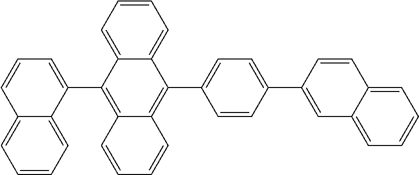 [SMILES: c1ccc2cc(-c3ccc(-c4c5ccccc5c(-c5cccc6ccccc56)c5ccccc45)cc3)ccc2c1]

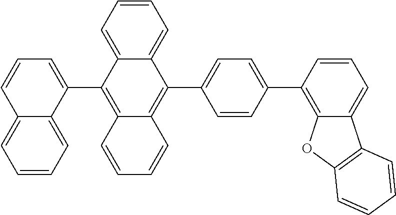 [SMILES: c1ccc2c(-c3c4ccccc4c(-c4ccc(-c5cccc6c5oc5ccccc56)cc4)c4ccccc34)cccc2c1]

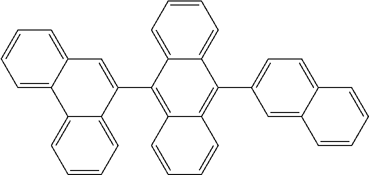 [SMILES: c1ccc2cc(-c3c4ccccc4c(-c4cc5ccccc5c5ccccc45)c4ccccc34)ccc2c1]

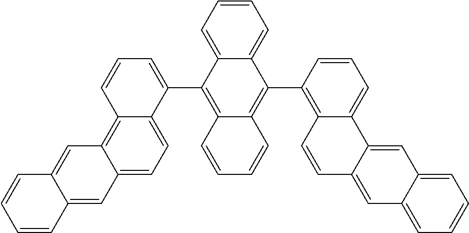 [SMILES: c1ccc2cc3c(ccc4c(-c5c6ccccc6c(-c6cccc7c6ccc6cc8ccccc8cc67)c6ccccc56)cccc43)cc2c1]

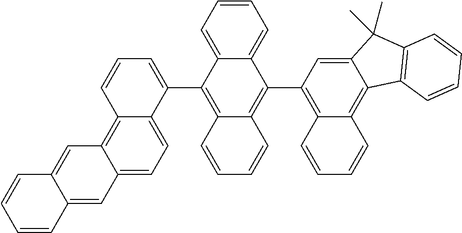 [SMILES: CC1(C)c2ccccc2-c2c1cc(-c1c3ccccc3c(-c3cccc4c3ccc3cc5ccccc5cc34)c3ccccc13)c1ccccc21]

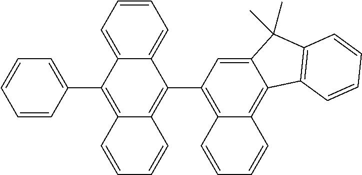 [SMILES: CC1(C)c2ccccc2-c2c1cc(-c1c3ccccc3c(-c3ccccc3)c3ccccc13)c1ccccc21]

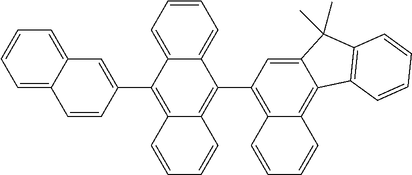 [SMILES: CC1(C)c2ccccc2-c2c1cc(-c1c3ccccc3c(-c3ccc4ccccc4c3)c3ccccc13)c1ccccc21]

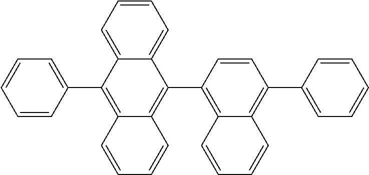 [SMILES: c1ccc(-c2ccc(-c3c4ccccc4c(-c4ccccc4)c4ccccc34)c3ccccc23)cc1]

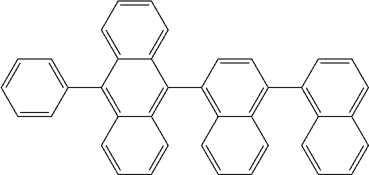 [SMILES: c1ccc(-c2c3ccccc3c(-c3ccc(-c4cccc5ccccc45)c4ccccc34)c3ccccc23)cc1]

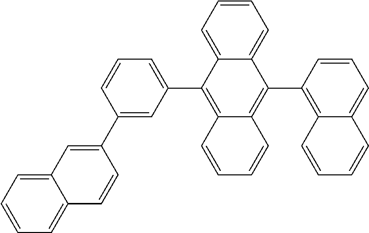 [SMILES: c1cc(-c2ccc3ccccc3c2)cc(-c2c3ccccc3c(-c3cccc4ccccc34)c3ccccc23)c1]

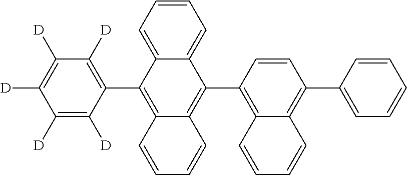 [SMILES: [2H]c1c([2H])c([2H])c(-c2c3ccccc3c(-c3ccc(-c4ccccc4)c4ccccc34)c3ccccc23)c([2H])c1[2H]]

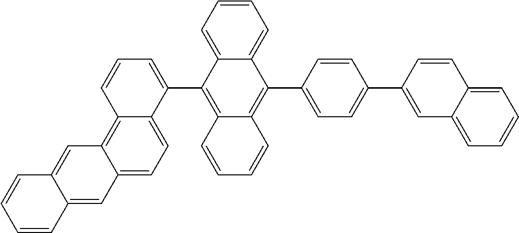 [SMILES: c1ccc2cc(-c3ccc(-c4c5ccccc5c(-c5cccc6c5ccc5cc7ccccc7cc56)c5ccccc45)cc3)ccc2c1]

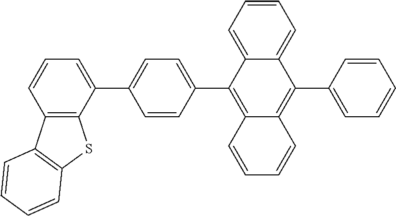 [SMILES: c1ccc(-c2c3ccccc3c(-c3ccc(-c4cccc5c4sc4ccccc45)cc3)c3ccccc23)cc1]

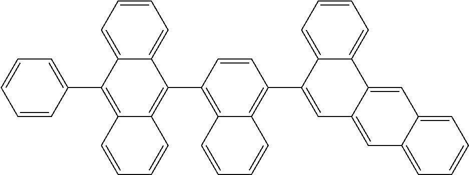 [SMILES: c1ccc(-c2c3ccccc3c(-c3ccc(-c4cc5cc6ccccc6cc5c5ccccc45)c4ccccc34)c3ccccc23)cc1]

Suitable charge transport materials as usable in the hole injection or hole transport layer or electron blocker layer or in the electron transport layer of the organic electroluminescent device of the invention are, as well as the compounds of the invention, for example, the compounds disclosed in Y. Shirota et al., Chem. Rev. 2007, 107(4), 953-1010, or other materials as used in these layers according to the prior art.

Examples of preferred hole transport materials which can be used in a hole transport, hole injection or electron blocker layer in the electroluminescent device of the invention are indenofluoreneamine derivatives (for example according to WO 06/122630 or WO 06/100896), the amine derivatives disclosed in EP 1661888, hexaazatriphenylene derivatives (for example according to WO 01/049806), amine derivatives having fused aromatic systems (for example according to U.S. Pat. No. 5,061,569), the amine derivatives disclosed in WO 95/09147, monobenzoindenofluoreneamines (for example according to WO 08/006449), dibenzoindenofluoreneamines (for example according to WO 07/140847), spirobifluoreneamines (for example according to WO 2012/034627 or WO 2013/120577), fluoreneamines (for example according to WO 2014/015937, WO 2014/015938 and WO 2014/015935), spirodibenzopyranamines (for example according to WO 2013/083216) and dihydroacridine derivatives (for example according to WO 2012/150001).

Preferred cathodes of the organic electroluminescent device are metals having a low work function, metal alloys or multilayer structures composed of various metals, for example alkaline earth metals, alkali metals, main group metals or lanthanoids (e.g. Ca, Ba, Mg, Al, In, Mg, Yb, Sm, etc.). Additionally suitable are alloys composed of an alkali metal or alkaline earth metal and silver, for example an alloy composed of magnesium and silver. In the case of multilayer structures, in addition to the metals mentioned, it is also possible to use further metals having a relatively high work function, for example Ag or Al, in which case combinations of the metals such as Ca/Ag, Mg/Ag or Ba/Ag, for example, are generally used. It may also be preferable to introduce a thin interlayer of a material having a high dielectric constant between a metallic cathode and the organic semiconductor. Examples of useful materials for this purpose are alkali metal or alkaline earth metal fluorides, but also the corresponding oxides or carbonates (e.g. LiF, Li₂O, BaF₂, MgO, NaF, CsF, Cs₂CO₃, etc.). It is also possible to use lithium quinolinate (LiQ) for this purpose. The layer thickness of this layer is preferably between 0.5 and 5 nm.

Preferred anodes are materials having a high work function. Preferably, the anode has a work function of greater than 4.5 eV versus vacuum. Firstly, metals having a high redox potential are suitable for this purpose, for example Ag, Pt or Au. Secondly, metal/metal oxide electrodes (e.g. Al/Ni/NiOₓ, Al/PtOₓ) may also be preferred. For some applications, at least one of the electrodes has to be transparent or partly transparent in order to enable the irradiation of the organic material (organic solar cell) or the emission of light (OLED, O-LASER). Preferred anode materials here are conductive mixed metal oxides. Particular preference is given to indium tin oxide (ITO) or indium zinc oxide (IZO). Preference is further given to conductive doped organic materials, especially conductive doped polymers.

The device is appropriately (according to the application) structured, contact-connected and finally sealed, since the lifetime of the devices of the invention is shortened in the presence of water and/or air.

In a preferred embodiment, the organic electroluminescent device of the invention is characterized in that one or more layers are coated by a sublimation process. In this case, the materials are applied by vapor deposition in vacuum sublimation systems at an initial pressure of less than 10⁻⁵ mbar, preferably less than 10⁻⁶ mbar. In this case, however, it is also possible that the initial pressure is even lower, for example less than 10⁻⁷ mbar.

Preference is likewise given to an organic electroluminescent device, characterized in that one or more layers are coated by the OVPD (organic vapor phase deposition) method or with the aid of a carrier gas sublimation. In this case, the materials are applied at a pressure between 10⁻⁵ mbar and 1 bar. A special case of this method is the OVJP (organic vapor jet printing) method, in which the materials are applied directly by a nozzle and thus structured (for example M. S. Arnold et al., Appl. Phys. Lett. 2008, 92, 053301).

Preference is additionally given to an organic electroluminescent device, characterized in that one or more layers are produced from solution, for example by spin-coating, or by any printing method, for example screen printing, flexographic printing, nozzle printing or offset printing, but more preferably LITI (light-induced thermal imaging, thermal transfer printing) or inkjet printing. For this purpose, soluble compounds of formula (I) are needed. High solubility can be achieved by suitable substitution of the compounds.

It is further preferable that an organic electroluminescent device of the invention is produced by applying one or more layers from solution and one or more layers by a sublimation method.

According to the invention, the electronic devices comprising one or more compounds of the invention can be used in displays, as light sources in lighting applications and as light sources in medical and/or cosmetic applications (e.g. light therapy).

### WORKING EXAMPLES

### A) Synthesis Examples

### A-1) Synthesis of Compound 1

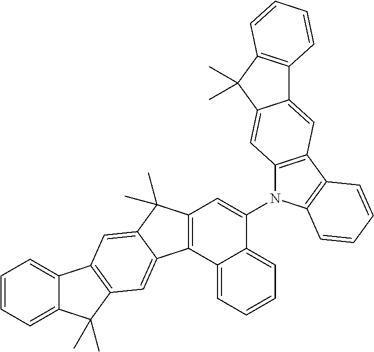 [SMILES: CC1(C)c2ccccc2-c2cc3c(cc21)-c1c(cc(-n2c4ccccc4c4cc5c(cc42)C(C)(C)c2ccccc2-5)c2ccccc12)C3(C)C]

5-Bromo-7,13-dihydro-7,7,13,13-tetramethylbenzo[g]indeno[1,2-b]fluorene (400 mg, 0.86 mmol, 95.4%), 5,7-dihydro-7,7-dimethylindeno-[2,1-b]carbazole (292.4 mg, 1.03 mmol, 1.2 equiv.), Pd₂(dba)₃ (16.07 mg, 0.017 mmol, 2 mol %) and SPhos (14.12 mg, 0.034 mmol, 4 mol %) are weighed into a vial, provided with protective gas atmosphere and sealed a septum, and 8 mL of toluene are added. Subsequently, at RT, K₃PO₄ (583.3 mg, 2.69 mmol, 3.1 equiv.) is added to the reaction mixture while stirring. The reaction mixture is heated overnight at 105° C. for 8 d in a heating block while stirring. After allowing it to cool to room temperature, distilled H₂O is added to the reaction solution and the aqueous phase is extracted with toluene. The organic phase is dried over MgSO₄ and concentrated, and the crude product is purified by column chromatography (eluent:heptane:DCM vol./vol. 15:1→1:1) on silica gel.

The product is obtained as a pale yellow solid (78 mg, 14%).

MS (El) m/z calculated for C₄₉H₃₉N: 641.3, found [M]+: 641.4.

Elemental analysis calculated (%) for C₄₉H₃₉N: C, 91.69, H, 6.12, N, 2.18, found: C, 91.62, H, 6.55, N, 2.07.

### A-2) Synthesis of Compound 2

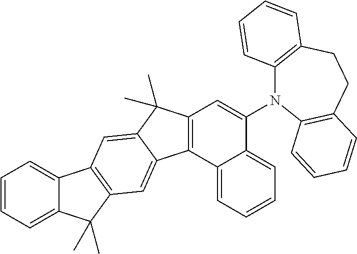 [SMILES: CC1(C)c2ccccc2-c2cc3c(cc21)-c1c(cc(N2c4ccccc4CCc4ccccc42)c2ccccc12)C3(C)C]

5-Bromo-7,13-dihydro-7,7,13,13-tetramethylbenzo[g]indeno[1,2-b]fluorene (400 mg, 0.86 mmol, 95.4%), 11-dihydro-5H-dibenz[b,f]azepine (184.7 mg, 0.95 mmol, 1.2 equiv.), Pd(OAc)₂ (3.93 mg, 0.017 mmol, 2 mol %) and SPhos (14.12 mg, 0.034 mmol, 4 mol %) are weighed into a vial, provided with protective gas atmosphere and sealed a septum, and 6 mL of toluene are added. Subsequently, n-hexyllithium (2.47 M in hexane) (0.39 mL, 0.96 mmol, 1.1 equiv.) is cautiously added dropwise to the reaction mixture at RT while stirring. The reaction mixture is heated overnight at 85° C. for one day in a heating block while stirring. After allowing it to cool to room temperature, distilled H₂O is added to the reaction solution and the aqueous phase is extracted with toluene. The organic phase is dried over MgSO₄ and concentrated, and the crude product is purified by column chromatography on silica gel (eluent:heptane:toluene vol./vol. 2:1→1:1→DCM).

The product is obtained as a pale yellow solid (28 mg, 6%).

MS (El) m/z calculated for C₄₂H₃₅N: 553.3, found [M]⁺: 553.3.

Elemental analysis calculated (%) for C₄₂H₃₅N: C, 91.10, H, 6.37, N, 2.53, found: C, 89.91, H, 7.18, N, 2.25.

### A-3) Synthesis of Compound 3

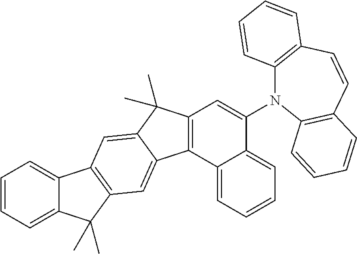 [SMILES: CC1(C)c2ccccc2-c2cc3c(cc21)-c1c(cc(N2c4ccccc4C=Cc4ccccc42)c2ccccc12)C3(C)C]

5-Bromo-7,13-dihydro-7,7,13,13-tetramethylbenzo[g]indeno[1,2-b]fluorene (400 mg, 0.86 mmol, 95.4%), 5H-dibenzo[b,f]azepine (199.5 mg, 1.03 mmol, 1.2 equiv.), Pd(OAc)₂ (3.93 mg, 0.017 mmol, 2 mol %) and SPhos (14.12 mg, 0.034 mmol, 4 mol %) are weighed into a vial, provided with protective gas atmosphere and sealed a septum, and 6 mL of toluene are added. Subsequently, n-hexyllithium (2.47 M in hexane) (0.39 mL, 0.96 mmol, 1.1 equiv.) is cautiously added dropwise to the reaction mixture at RT while stirring. The reaction mixture is heated overnight at 85° C. for one day in a heating block while stirring. After allowing it to cool to room temperature, distilled H₂O is added to the reaction solution and the aqueous phase is extracted with toluene. The organic phase is dried over MgSO₄, filtered (under basic conditions) through AlOx and concentrated. The residue obtained is treated with acetonitrile and 2-propanol, and the precipitated solids are filtered and dried under reduced pressure. 445 mg (93%) of the product are obtained in the form of a shiny yellow solid.

MS (El) m/z calculated for C₄₂H₃₃N: 551.3, found [M]⁺: 551.4.

Elemental analysis calculated (%) for C₄₂H₃₃N: C, 91.43, H, 6.03, N, 2.54; found: C, 91.13, H, 6.10, N, 2.52.

### B) Device Examples

### Production of the OLEDs

OLEDs of the invention and OLEDs according to the prior art are produced by a general method according to WO 04/058911, which is adapted to the circumstances described here (variation in layer thickness, materials).

In the examples which follow (see tables 1 to 3), the data of various OLEDs are presented. Substrates used are glass substrates coated with structured ITO (indium tin oxide) of thickness 50 nm. The OLEDs basically have the following layer structure: substrate/buffer/hole injection layer 1 (95% HIL1+5% HIL2, 20 nm)/hole transport layer (HTL, thickness stated in table 1)/emission layer (EML, 20 nm)/electron transport layer (50% ETL+50% EIL, 20 nm)/electron injection layer (EIL, 3 nm) and finally a cathode. The cathode is formed by an aluminum layer of thickness 100 nm. The buffer applied by spin-coating is a 20 nm-thick layer of Clevios P VP Al 4083 (sourced from Heraeus Clevios GmbH, Leverkusen). All the rest of the materials are applied by thermal vapor deposition in a vacuum chamber. The structure of the OLEDs is shown in table 1. The materials used are shown in table 3.

The emission layer (EML) always consists of at least one matrix material (host, H) and an emitting dopant (D) which is added to the matrix material in a particular proportion by volume by co-evaporation. Details given in such a form as H1:D1 (97%:3%) mean here that the material H1 is present in the layer in a proportion by volume of 97% and D1 in a proportion by volume of 3%.

The OLEDs are characterized in a standard manner. For this purpose, the electroluminescence spectra are recorded, and the current efficiency (measured in cd/A) and the external quantum efficiency (EQE, measured in percent) are calculated as a function of luminance, assuming Lambertian emission characteristics, from current-voltage-luminance characteristics (IUL characteristics), and finally the lifetime of the components is determined. The electroluminescence spectra are recorded at a luminance of 1000 cd/m², and the CIE 1931 x and y color coordinates are calculated therefrom. The parameter EQE @ 10 mA/cm² refers to the external quantum efficiency at an operating current density of 10 mA/cm². The lifetime LD95 @ 10 mA/cm² is the time that passes before the starting brightness at an operating current density of 10 mA/cm² has dropped by 5%. The data obtained for the various OLEDs are collated in table 2.

Results: Use of the Compounds of the Invention as Dopants in Fluorescent OLEDs

The compounds of the invention are particularly suitable as blue-fluorescing dopants. The inventive compound D2 is used in the present examples as emitter in the emitting layer of OLEDs, in each case in combination with one of the host materials H1 and H2. As a comparative example, the emitter C-D1 is analyzed, likewise in each case in combination with one of the host materials H1 and H2.

The inventive OLEDs obtained are identified as 13 and 14 in table 2. They exhibit very good lifetime with deep blue emission. Compared to the emitter material C-D1 known in the prior art (cf. OLEDs C1 and C2 in table 2), both the external quantum efficiency and the lifetime are significantly improved, with deep blue emission.

TABLE 1
Structure of the OLEDs

| | | EML |
|---|---|---|
| Ex. | HTL | Thickness/nm |
| C1 | 20 nm | H1 (95%):C-D1 (5%)/20 nm |
| C2 | 195 nm | H2 (95%):C-D1 (5%)/20 nm |
| I3 | 20 nm | H1 (95%):D2 (5%)/20 nm |
| I4 | 195 nm | H2 (95%):D2 (5%)/20 nm |

TABLE 2
Data of the OLEDs

| | | |
|---|---|---|
| EQE @ | LD95 @ | |
| 10 mA/cm² | 10 mA/cm² | CIE |

| Ex. | % | [h] | x | y |
|---|---|---|---|---|
| C1 | 6.8 | 55 | 0.149 | 0.123 |
| C2 | 6.5 | 50 | 0.149 | 0.115 |
| I3 | 7.5 | 90 | 0.150 | 0.118 |
| I4 | 7.2 | 80 | 0.149 | 0.121 |

TABLE 3
Structures of the materials used

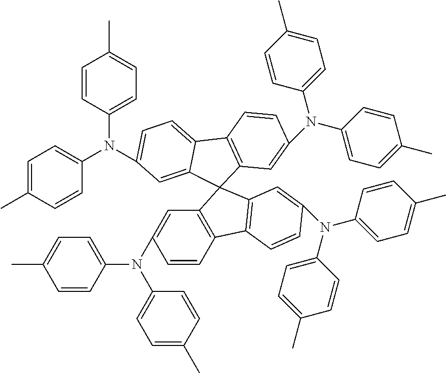

HIL1

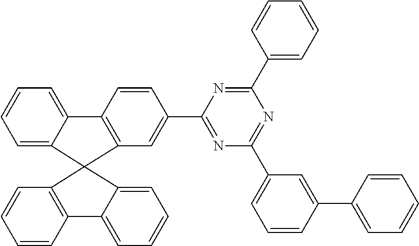 [SMILES: c1ccc(-c2cccc(-c3nc(-c4ccccc4)nc(-c4ccc5c(c4)C4(c6ccccc6-c6ccccc64)c4ccccc4-5)n3)c2)cc1]

ETL

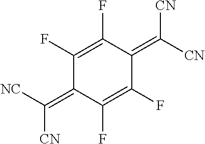 [SMILES: N#CC(C#N)=c1c(F)c(F)c(=C(C#N)C#N)c(F)c1F]

HIL2

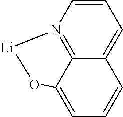

EIL

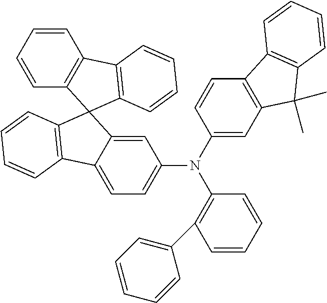 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

HTL

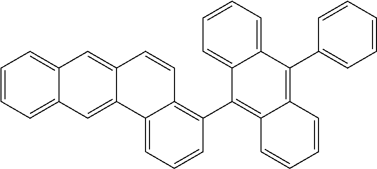 [SMILES: c1ccc(-c2c3ccccc3c(-c3cccc4c3ccc3cc5ccccc5cc34)c3ccccc23)cc1]

H1

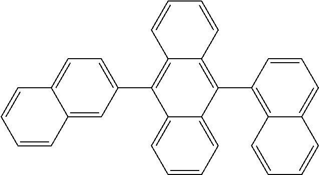 [SMILES: c1ccc2cc(-c3c4ccccc4c(-c4cccc5ccccc45)c4ccccc34)ccc2c1]

H2

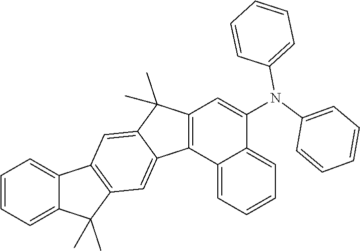 [SMILES: CC1(C)c2ccccc2-c2cc3c(cc21)-c1c(cc(N(c2ccccc2)c2ccccc2)c2ccccc12)C3(C)C]

C-D1

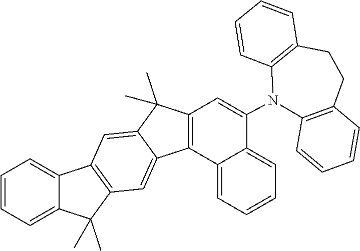 [SMILES: CC1(C)c2ccccc2-c2cc3c(cc21)-c1c(cc(N2c4ccccc4CCc4ccccc42)c2ccccc12)C3(C)C]

D2

1-18. (canceled)

19. A compound of formula (I): 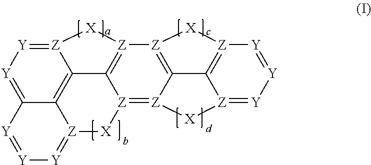
wherein
Y is the same or different in each instance and is CR¹ or N;
Z is C if X is present and is Y if X is not present;
X is the same or different in each instance and is a bivalent group selected from the group consisting of C(R¹)₂ and Si(R¹)₂;
R¹ is the same or different in each instance and is selected from the group consisting of H, D, F, C(═O)R², CN, Si(R²)₃, N(R²)₂, P(═O)(R²)₂, OR², S(═O)R², S(═O)₂R², straight-chain alkyl, or alkoxy groups having 1 to 20 carbon atoms, branched or cyclic alkyl or alkoxy groups baying 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; wherein two or more radicals R¹ are optionally joined to one another so as to define a ring; wherein the alkyl, alkoxy, alkenyl, and all ynyl groups, and the aromatic and heteroaromatic ring systems are each optionally substituted by one or more radicals R²; and wherein one or more CH₂ groups in the alkyl, alkoxy, alkenyl, and alkenyl groups are optionally replaced by —R²C═CR²—, —C≡C—, Si(R²)₂, C═O, C═NR², —C(═O)O—, C(═O)NR²—, NR², P(═O)(R²), —O—, —S—, SO, or SO₂;
R² is the same or different in each instance and is selected from the group consisting of H, D, CN, alkyl groups having 1 to 20 carbon atoms, aromatic ring systems having 6 to 40 aromatic ring atoms, and heteroaromatic ring systems having 5 to 40 aromatic ring atoms; wherein two or more radicals R² are optionally joined to one another so as to define a ring; and wherein the alkyl groups, aromatic ring systems, and heteroaromatic ring systems are each optionally substituted by F CN;
a, b, c, and d
are the same or different in each instance and are 0 or 1, with the proviso that a+b=1 and c+d=1, wherein when any one of a, b, c, or d is 0, the corresponding X is absent;
wherein at least one Y has a group of formula (N) bonded thereto instead of R¹, 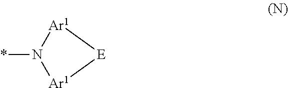
wherein
the bond identified by an asterisk indicates the bond to Y;
Ar¹ is the same or different in each instance and is selected from the group consisting of aromatic ring systems having 6 to 30 aromatic ring atoms and optionally substituted by one or more radicals R¹ and heteroaromatic ring systems which have 5 to 30 aromatic ring atoms and optionally substituted by one or more radicals R¹;
E is a single bond or a divalent group selected from the group consisting of arylene groups optionally substituted by one or more radicals R¹, heteroarylene groups optionally substituted by one or more radicals R¹, B(R¹) C(R¹)₂, C(R¹)═C(R¹), C≡C, Si(R¹)₂; C═O, C═NR¹, C═C(R¹)₂, O, S, S═O, SO₂, N(R¹), P(R¹), and P(═O)R¹, combinations of 2, 3, or 4 identical or different groups among these.

20. The compound of claim 19, wherein the compound of formula, (1) contains exactly one Y wherein a group of formula (N) is bonded thereto instead of R¹.

21. The compound of claim 19, wherein in the group of formula (N), Ar¹ is the same or different in each instance and is selected from the group consisting of phenyl, naphthyl, phenanthrenyl, biphenyl, terphenyl, quaterphenyl, fluorenyl, indenofluorenyl, carbazolyl, dibenzothiophenyl, dibenzofusanyl, benzofuranyl, benzothiophenyl, indolyl, triazinyl, pyrimidinyl, pyridyl, and pyridazinyl, each of which is optionally substituted by one or more radicals R¹.

22. The compound of claim 19, wherein Ar¹ is the same in each instance.

23. The compound of claim 19, wherein E in the group of formula (N) is the same or different in each instance and is selected from the group consisting of a single bond, arylene groups optionally substituted by one or more radicals R¹, heteroarylene groups optionally substituted by one or more radicals R¹, and the following divalent groups;

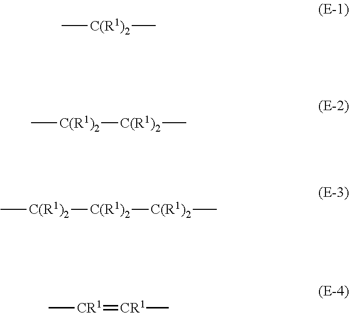

24. The compound of claim 19, wherein the group of formula (N) is a group of formulae (N-1) through (N-12): 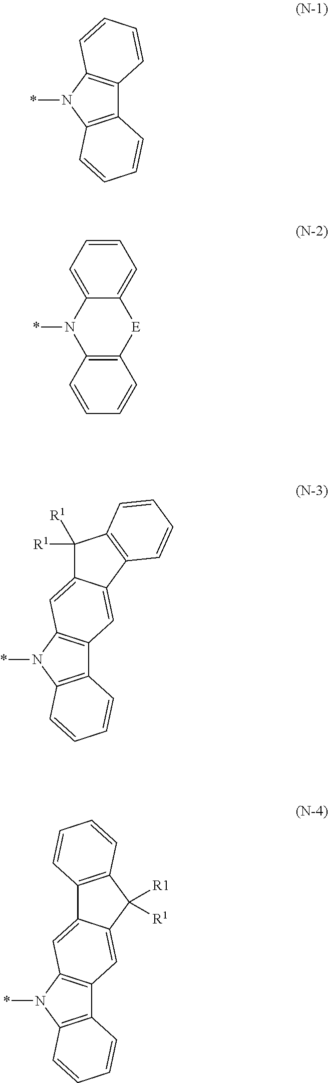 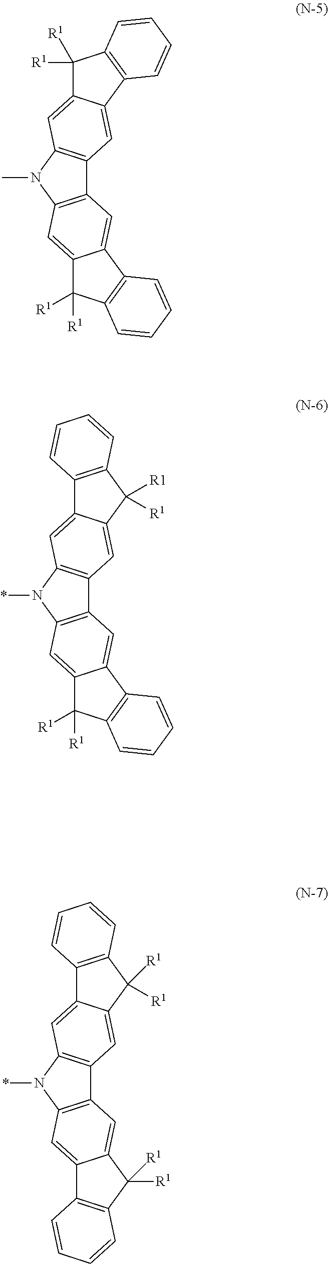 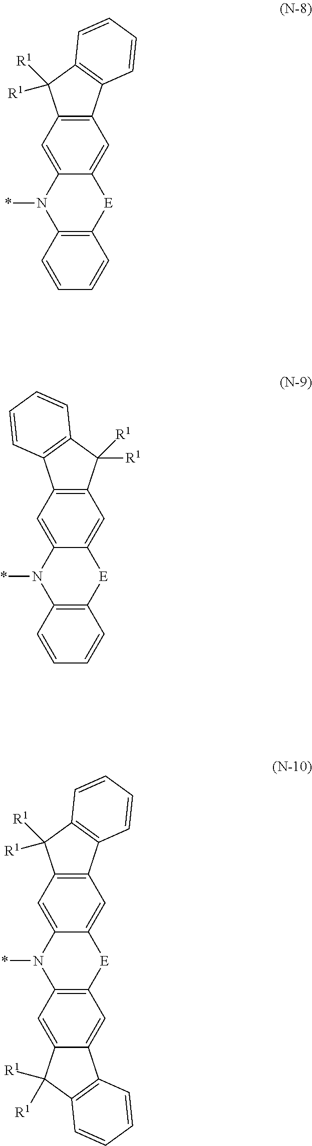 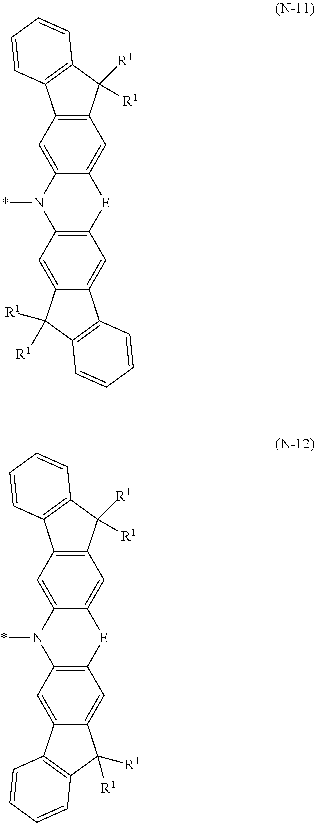
wherein the groups of formulae (N-1) through (N-12) is optionally substituted with a radical R¹ at any position indicated as unsubstituted.

25. The compound of claim 19, wherein all Y are CR¹.

26. The compound of claim 19, wherein X is C(R¹)₂.

27. The compound of claim 19, wherein the radicals R¹ of X are the same or different in each instance and are selected from the group consisting of straight-chain alkyl groups having 1 to 12 carbon atoms, branched or cyclic alkyl groups having 3 to 12 carbon atoms, and aromatic ring systems having 6 to 20 aromatic ring atoms; wherein the alkyl groups and aromatic ring systems are each optionally substituted by one of more radicals R².

28. The compound of claim 19, Therein the radicals R¹ in Y are the same or different each instance and are selected from the group consisting of H, D, F, CN, straight-chain alkyl groups having 1 to 20 carbon atoms, branched or cyclic alkyl groups having 3 to 20 carbon atoms, aromatic ring systems having 6 to 40 aromatic ring atoms, and heteroaromatic ring systems having 5 to 40 aromatic ring atoms, wherein the alkyl groups and aromatic and heteroaromatic ring systems are each optionally substituted by one or more radicals R².

29. The compound of claim 19, wherein the compound of formula (I) is a compound of formulae (I-1-1) through (I-4-1):

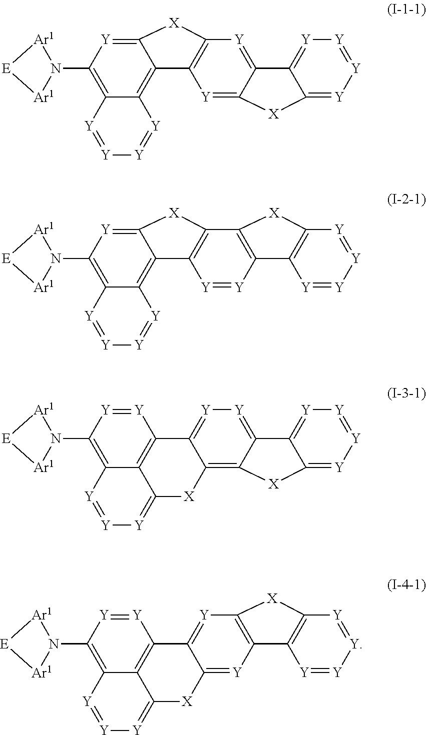

30. A process for preparing the compound of formula (I) of claim 19, comprising reacting a monobenzoindenofluorene derivative comprising one or more reactive groups with an amine in a transition pleat-catalyzed coupling reaction.

31. An oligomer, polymer, or dendrimer comprising one or more compounds of formula (I) of claim 19, wherein the bond(s) to the polymer, oligomer, or dendrimer are located at any desired position(s) substituted by R¹ or R².

32. A formulation comprising at least one compound of formula (I) of claim 19 and at least one solvent.

33. A formulation comprising at, least one oligomer, polymer, or dendrimer of claim 31 and at least one solvent.

34. An electronic device comprising at least one compound of formula (I) of claim 19.

35. An electronic device comprising at least one oligomer, polymer, or dendrimer of claim 31.

36. The electronic device of claim 34, wherein the electronic device is an organic electroluminescent device comprising an anode, a cathode and at least one emitting layer, wherein at least one organic layer of the device, which is optionally an emitting layer, a hole transport layer, or another layer, comprises the at least one compound.

37. The electronic device of claim 35, wherein the electronic device is an organic electroluminescent device comprising an anode, a cathode and at least one emitting layer, wherein at least one organic layer of the device, which is optionally an emitting layer, a hole transport layer, or another layer, comprises the at least one oligomer, polymer, or dendrimer.

38. The electronic device of 36, wherein the at least one organic layer is an emitting layer.

39. The electronic device of 37, wherein the at least one organic layer is an emitting layer.

40. The electronic device of 38, wherein the emitting layer further comprises one or more matrix compounds.

41. The electronic device of 39, wherein the emitting layer further comprises one or more matrix compounds.
